(19) United States
(12) Patent Application Publication
(10) Pub. No.: US 2025/0000784 A1
(43) Pub. Date: Jan. 2, 2025

(54) PERSONAL CARE COMPOSITIONS HAVING ENHANCED ANTIOXIDANT PERFORMANCE

(71) Applicant: Edgewell Personal Care Brands, LLC, Chesterfield, MO (US)

(72) Inventors: Diana Rojas, Avenel, NJ (US); Kamil Wojtowicz, Allendale, NJ (US); Kelly Ruffenach, Cedar Grove, NJ (US); Michael Russell, Madison, NJ (US); Samuel Vona, Keene Valley, NY (US); Smaranda Savarino, Stockholm, NJ (US)

(21) Appl. No.: 18/694,533

(22) PCT Filed: Sep. 23, 2022
(86) PCT No.: PCT/US2022/076900

§ 371 Date: Mar. 22, 2024

Related U.S. Application Data
(60) Provisional application No. 63/247,490, filed on Sep. 23, 2021.

(51) Int. Cl. A61K 8/9789; A61K 8/06; A61K 8/67; A61K 8/73; A61K 8/81; A61Q 17/04

(52) U.S. Cl. CPC A61K 8/9789; A61K 8/062; A61K 8/676; A61K 8/73; A61K 8/8147; A61Q 17/04; A61K 2800/48

(57) ABSTRACT

Embodiments relate to personal care compositions including Camellia sinensis (green tea) extract present in an effective amount to provide enhanced antioxidant power. The Camellia sinensis (green tea) extract may be in crystalline dosage form. The Camellia sinensis (green tea) extract may be initially dispersed in an aqueous solvent to provide an aqueous dosage form. The Camellia sinensis (green tea) extract may be initially dispersed in a non-aqueous anhydrous solvent to provide a non-aqueous dosage form. The personal care composition may further include a sunscreen filter. The personal care composition may further include a vitamin or a derivative thereof.

### CROSS-REFERENCE TO RELATED APPLICATIONS

This application claims priority to U.S. Provisional Patent Application No. 63/247,490, filed on Sep. 23, 2021, the contents of which is incorporated herein by reference in its entirety.

### TECHNICAL FIELD

Embodiments relate to personal care compositions including Camellia sinensis (green tea) extract in an effective amount to provide enhanced antioxidant performance.

### BACKGROUND

Technologies are available to protect a keratinous substance from damaging effects of electromagnetic radiation. For example, personal care compositions may include sunscreen filters to provide photoprotection by absorbing or scattering ultraviolet (UV) radiation. Personal care compositions may also include antioxidants to address damaging effects caused by free radicals formed from UV radiation incident on a keratinous substance.

Antioxidants, however, may be used in arbitrary amounts without formulation or manufacturing considerations that can dampen performance or unnecessarily waste the antioxidants. Thus, there is room for improvement to provide personal care compositions including antioxidants in effective amounts to provide enhanced antioxidant performance.

### SUMMARY

Embodiments relate to a personal care composition including Camellia sinensis (green tea) extract present in an effective amount based at least in part on formulation format, manufacturing process, dosage form, or combinations thereof. Notably, embodiments provide enhanced antioxidant performance and/or minimize waste of costly ingredients.

Embodiments further relate to a personal care composition including water present in an amount from 50.0 wt. % to 90.0 wt. %, based on the total weight of the composition, an emulsifier having a hydrophilic-lipophilic balance (HLB) value 7, an un-neutralized polymer thickener present in an amount from 0.1 wt. % to 1.0 wt. %, based on the total weight of the composition, and Camellia sinensis (green tea) extract present in an amount from 0.005 wt. % to 0.2 wt. %, based on the total weight of the composition. The Camellia sinensis (green tea) extract may be initially dispersed in an aqueous solvent to provide an aqueous dosage form. In one example, the un-neutralized polymer thickener may include xanthan gum, the Camellia sinensis (green tea) extract may be present in an amount from 0.1 wt. % to 0.2 wt. %, based on the total weight of the composition, and the personal care composition may exclude any sunscreen filter. The personal care composition may further include a sunscreen filter. For example, the sunscreen filter may include an organic sunscreen filter, a mineral sunscreen filter, or combinations thereof, the un-neutralized polymer thickener may include xanthan gum, and the Camellia sinensis (green tea) extract may be present in an amount from 0.1 wt. % to 0.2 wt. %, based on the total weight of the composition.

Embodiments further relate to a personal care composition Including water present in an amount from 50.0 wt. % to 90.0 wt. %, based on the total weight of the composition, an emulsifier having a hydrophilic-lipophilic balance (HLB) value≥7, a neutralized polymer thickener present in an amount from 0.1 wt. % to 1.0 wt. %, based on the total weight of the composition, and Camellia sinensis (green tea) extract present in an amount from 0.04 wt. % to 0.2 wt. %, based on the total weight of the composition. The Camellia sinensis (green tea) extract may be initially dispersed in an aqueous solvent to provide an aqueous dosage form. In one example, the neutralized polymer thickener may include carbomer, and the Camellia sinensis (green tea) extract may be present in an amount from 0.1 wt. % to 0.2 wt. %, based on the total weight of the composition. The personal care composition may further include a sunscreen filter. For example, the sunscreen filter may include an organic sunscreen filter, the neutralized polymer thickener may include acrylates/C₁₀-C₃₀alkyl acrylate crosspolymer, and the Camellia sinensis (green tea) extract may be present in an amount from 0.1 wt. % to 0.2 wt. %, based on the total weight of the composition.

In any of the preceding or foregoing embodiments, the personal care composition may further include a vitamin C derivative. For example, a ratio of the Camellia sinensis (green tea) extract to the vitamin C derivative may be from about 1:1 to about 1:20. For example, the vitamin C derivative may include ascorbyl glucoside. Thus, for example, the sunscreen filter may include an organic sunscreen filter, the neutralized polymer thickener may include carbomer, and the Camellia sinensis (green tea) extract may be present in an amount from 0.04 wt. % to <0.1 wt. %, based on the total weight of the composition.

Embodiments further relate to a personal care composition including water present in an amount from 10.0 wt. % to <50.0 wt. %, based on the total weight of the composition, an emulsifier having a hydrophilic-lipophilic balance (HLB) value≤6, and Camellia sinensis (green tea) extract present in an amount from 0.05 wt. % to 0.5 wt. %, based on the total weight of the composition. The personal care composition may further include a sunscreen filter. The personal care composition may further include ascorbic acid or a derivative thereof.

In any of the preceding or foregoing embodiments, the Camellia sinensis (green tea) extract may be initially dispersed in a non-aqueous anhydrous solvent to provide a non-aqueous dosage form. In one example, the Camellia sinensis (green tea) extract may be initially dispersed in a non-aqueous anhydrous solvent and the Camellia sinensis (green tea) extract may be present in an amount from 0.05 wt. % to 0.2 wt. %, based on the total weight of the composition. In another example, the Camellia sinensis (green tea) extract may be initially dispersed in a C₂-C₄ alcohol, a hydrocarbon, or combinations thereof, and the Camellia sinensis (green tea) leaf extract may be present in an amount from 0.01 wt. % to 0.15 wt. %, based on the total weight of the composition.

In any of the preceding or foregoing embodiments, the Camellia sinensis (green tea) extract may be initially dispersed in an aqueous solvent to provide an aqueous dosage form. In addition, the Camellia sinensis (green tea) extract in aqueous dosage form may be further dispersed in a non-aqueous anhydrous solvent. For example, the Camellia sinensis (green tea) extract in aqueous dosage form may be further dispersed a C₂-C₄ alcohol, and the Camellia sinensis (green tea) extract may be present in an amount from 0.05 wt. % to 0.3 wt. %, based on the total weight of the composition. In addition, the Camellia sinensis (green tea) extract in aqueous dosage form may be further dispersed in a hydrocarbon. For example, the Camellia sinensis (green tea) extract in aqueous dosage form may be further dispersed in a hydrocarbon, and the Camellia sinensis (green tea) extract may be present in an amount from 0.2 wt. % to 0.3 wt. %, based on the total weight of the composition.

Embodiments further relate to a personal care composition including water present in an amount ≤5.0 wt. %, based on the total weight of the composition, an un-neutralized wax thickener present in an amount from 15.0 wt. % to 30.0 wt. %, based on the total weight of the composition, and Camellia sinensis (green tea) extract present in an amount from 0.005 wt. % to 0.2 wt. %, based on the total weight of the composition. The un-neutralized wax thickener may be selected from an animal wax, a vegetable wax, a mineral wax, a petroleum wax, a silicone wax, or combinations thereof. The Camellia sinensis (green tea) extract may be in crystalline dosage form. The personal care composition may further include a sunscreen filter. For example, the Camellia sinensis (green tea) extract may be present in an amount from 0.05 wt. % to 0.2 wt. %, based on the total weight of the composition, and the un-neutralized wax thickener may be present in an amount from 18.0 wt. % to 25.0 wt. %, based on the total weight of the composition.

Embodiments further relate to a personal care composition including water present in an amount ≤5.0 wt. %, based on the total weight of the composition, a C₂-C₄ alcohol solvent present in an amount from 35.0 wt. % to 80.0 wt. %, based on the total weight of the composition, and Camellia sinensis (green tea) extract present in an amount from 0.005 wt. % to <0.1 wt. %, based on the total weight of the composition. The Camellia sinensis (green tea) extract may be in crystalline dosage form. The personal care composition may further include a sunscreen filter. In one example, the Camellia sinensis (green tea) extract may be present in an amount from 0.05 wt. % to <0.1 wt. %, based on the total weight of the composition, and the personal care composition may further include a propellant. In another example, the Camellia sinensis (green tea) extract may be present in an amount from 0.01 wt. % to 0.05 wt. %, based on the total weight of the composition. The personal care composition may further include a vitamin C derivative. The vitamin C derivative may include tetrahexyldecyl ascorbate.

Embodiments further relate to a personal care composition including water present in an amount ≤5.0 wt. %, based on the total weight of the composition, a non-polar carrier present in an amount from 20.0 wt. % to 80.0 wt. %, based on the total weight of the composition, and Camellia sinensis (green tea) extract present in an amount from 0.005 wt. % to 0.2 wt. %, based on the total weight of the composition. The Camellia sinensis (green tea) extract may be present in an amount from 0.05 wt. % to 1.0 wt. %, based on the total weight of the composition. The non-polar carrier may be selected from an ester, an ether, a fatty alcohol, an isoparaffin, a silicone oil, a hydrocarbon oil, a polyol, or combinations thereof. The personal care composition may further include a C₂-C₄ alcohol solvent.

In any of the preceding or foregoing embodiments, the personal care composition may further include one or more film formers, anti-aging actives, insect repellants, amino acids, colorants, pigments, dyes, opacifiers, pH adjusters, fragrances, fillers, vitamins and its derivatives, preservatives, preservative boosters, chelating agents, stabilizers, sphingolipids, antioxidants, minerals, keratolytics, hormonal compounds, analgesics, anti-allergenic agents, H1 or H2 antihistamines, anti-inflammatory agents, anti-irritants, anti-neoplastics, immune system boosting agents, immune system suppressing agents, anti-acne agents, anesthetics, antiseptics, skin cooling compounds, skin protectants, skin penetration enhancers, exfoliants, staining agents, depigmenting agents, hypopigmenting agents, stabilizers, pharmaceutical agents, spherical powders, extracts, alpha and beta hydroxy acids, retinol and its derivatives, elastomers, propellants, or combinations thereof.

Embodiments further relate to a method to protect a keratinous surface (e.g., from UV radiation) including contacting the keratinous surface with the personal care composition of any of the preceding embodiments. Embodiments further relate to a method to manufacture a personal care composition of any the preceding embodiments, comprising at least adding Camellia sinensis (green tea) in an effective amount to provide enhanced antioxidant performance. Embodiments further relate to a consumer packaged product including the personal care composition of any of the preceding embodiments.

### DETAILED DESCRIPTION

Embodiments can comprise, consist of, and consist essentially of the features and/or steps described herein, as well as any of the additional or optional ingredients, components, steps, or features described herein or would otherwise be appreciated by one of skill in the art. It is to be understood that all concentrations disclosed herein are by weight percent (wt. %.) based on a total weight of the composition unless otherwise indicated. Where appropriate, the International Nomenclature of Cosmetic Ingredients (INCI) name of ingredients/components is provided. Any numerical range recited herein is intended to include all sub-ranges subsumed therein, and such ranges are understood to include each and every number and/or fraction between the stated range lower and upper values. Moreover, the term “about” and the like may refer to deviations of ±20% unless otherwise stated.

### Antioxidant(s)

Personal care compositions include antioxidants to counter free radicals that damage biomolecules. Antioxidants may be obtained, for example, from the extracts of flowers, fruits, vegetables, and plants. Thus, personal care compositions may include Camellia sinensis (green tea) extract, Daucus carota sativa (carrot) root extract, or combinations thereof. Camellia sinensis (green tea) extract, which may be concentrate extracted from green tea leaves (INCI: Camellia Sinensis Leaf Extract), can contain various polyphenols and catechin derivatives such as catechin, epigallocatechin, epicatechin, epicatechin gallate, epigallocatechin gallate, gallocatechin gallate, gallocatechin, or combinations thereof. Such constituents are effective scavengers of reactive oxygen species and may also function indirectly as antioxidants through their effects on transcription factors and enzyme activities.

Camellia sinensis (green tea) extract is commercially available for use in cosmetic or pharmaceutical compositions. Camellia sinensis (green tea) extract may be commercially available in a crystalline dosage form (e.g., powder form). For example, one commercially available Camellia sinensis (green tea) extract in crystalline dosage form retains≥98% total phenols, ≥80% total catechins, and 45% total epigallocatechin gallate. While Camellia sinensis (green tea) extract in crystalline dosage form may be directly added into a formulation during the manufacture of a personal care composition, it may be preferable to initially disperse the extract in one or more solvents (dispersants).

Accordingly, Camellia sinensis (green tea) extract in crystalline dosage form may initially be dispersed in a non-aqueous solvent such as, without limitation, an alcohol, an ether, a fatty acid, a silicone fluid, an ester, a hydrocarbon, a fluorinated hydrocarbon, or combinations thereof. For example, Camellia sinensis (green tea) extract in crystalline dosage form may first be dispersed in adipates (e.g., diisopropyl adipate), paraffins or isoparaffins (e.g., C₁₃-C₁₆ isoparaffins such as isododecane and isohexadacane), alcohols (e.g., C₂-C₄ alcohols such as ethanol, etc.), organosilicon compounds (e.g., disiloxane, dimethicone, cyclopentasiloxane, etc.), or combinations thereof. Preferably, a premix is formed by dispersing Camellia sinensis (green tea) extract (in crystalline dosage form) into a non-aqueous solvent such as isododecane, ethanol, or combinations thereof. In one example, a ratio of Camellia sinensis (green tea) extract to non-aqueous solvent may be 1:5 to 1:15, preferably 1:10. For example, the ratio by weight of the Camellia sinensis (green tea) extract to non-aqueous solvent may be 1:5, 1:6, 1:7, 1:8, 1:9, 1:10, 1:11, 1:12, 1:13, 1:14, 1:15, including every number and/or fraction there between. Camellia sinensis (green tea) extract in a non-aqueous dosage form (premix) may then be added to a formulation phase during manufacture of a personal care composition or at the end when all phases have been combined (e.g., at relatively low temperature such as 40° C.).

Camellia sinensis (green tea) extract may also be commercially available in an aqueous dosage form (premix). Camellia sinensis (green tea) extract may, for example, first be dispersed in an aqueous solvent to form the aqueous dosage form. For example, Camellia sinensis (green tea) extract in aqueous dosage form may be composed of Camellia sinensis (green tea) extract present in a mixture (e.g., premix) with one or more other ingredients such as, without limitation, water, a diol (e.g., propylene glycol), additive(s) (e.g., sorbitol, citric, ascorbic acid, phenoxyethanol, sodium hydroxide ethylhexyl glycerin, etc.), or combinations thereof. Thus, for example, a ratio of Camellia sinensis (green tea) extract to dispersant may be about 1:1 to about 1:10. For example, the ratio by weight of the Camellia sinensis (green tea) extract to dispersant may be 0.8:12, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, 1:10, 1:10.5, 1:11, 1:11.5, 1:12, including every number and/or fraction there between. Camellia sinensis (green tea) extract in aqueous dosage form (premix) may then be further mixed with a non-aqueous solvent and/or may then be added to a formulation phase during the manufacture of a personal care composition or at the end when all phases have been combined (e.g., at relatively low temperature).

Personal care compositions may include additional antioxidants such as, for example, carotenoids (e.g., beta-carotene, lycopene, lutein, zeaxanthin), additional polyphenols (e.g., resveratrol), selenium, vitamins, derivatives thereof, or combinations thereof. For example, a vitamin may be selected from vitamin A (retinoic acid), vitamin B (e.g., nicotinic acid or nicotinamide), vitamin C (ascorbic acid), vitamin E (tocopherol), or combinations thereof. Meanwhile, a vitamin derivative may be selected from esters of vitamin A (e.g., retinyl acetate, etc.), vitamin C (ascorbyl palmitate, etc.), vitamin E (e.g., tocopheryl acetate, tocopheryl palmitate, tocopheryl linoleate, etc.), or combinations thereof. Thus, personal care compositions may include a vitamin and a derivative thereof, two or more derivatives of a vitamin, two or more different vitamins, two or more different derivatives of different vitamins, esters thereof (e.g., tocopheryl nicotinate), and so on. In one example, personal care compositions include a vitamin C derivative such as ascorbyl glucoside, tetrahexyldecyl ascorbate, or combinations thereof. One or more additional antioxidants may also be added to a formulation in crystalline dosage form or may be initially dispersed in an aqueous solvent such as water and/or in a non-aqueous solvent to form a premix.

Antioxidants may be present from about 0.0001 wt. % to about 1.0 wt. %, based on the total weight of the personal care composition. For example, Camellia sinensis (green tea) extract may be present in an amount of 0.005 wt. %, 0.01 wt. %, 0.045, 0.098, 0.15 wt. %, 0.2 wt. %, 0.3 wt. %, 0.5 wt. %, 0.99 wt. %, including every number and/or fraction therebetween, based on the total weight of the personal care composition. The amount of Camellia sinensis (green tea) extract used, with or without a premix, and the amount of premix used to achieve a desired amount extract in the personal care composition, can be readily calculated. Similarly, one or more additional antioxidants such as Daucus carota sativa (carrot) root extract or a vitamin C derivative may be present in an amount of 0.0001 wt. %, 0.001 wt. %, 0.005 wt. %, 0.01 wt. %, 0.045, 0.098, 0.15 wt. %, 0.2 wt. %, 0.3 wt. %, 0.5 wt. %, including every number and/or fraction there between, based on the total weight of the personal care composition, which can similarly be readily calculated based on a dosage form used.

### Thickener(s)

Personal care compositions may include a thickener to alter viscosity. Examples of thickeners include plant-based polymer thickeners, mineral thickeners, wax thickeners, synthetic polymer thickeners, or combinations thereof. Plant-based polymer thickeners may include, without limitation, xanthan gum, konjac gum, guar gum, acacia gum, cellulose derivatives such as hydroxycellulose, hydroxyethyl cellulose, or combinations thereof. Mineral thickeners may include, without limitation, silica, bentonite, hectorite, magnesium aluminum silicate, or combinations thereof. Natural or synthetic waxes may include, without limitation, animal waxes, vegetable waxes, mineral waxes, petroleum waxes, silicone waxes, or combinations thereof.

Examples of useful wax thickeners include, without limitation, alkyl silicone, alkyl trimethylsilane, beeswax, behenyl behenate, cerotyl dimethicone, candelilla wax, carnauba wax, synthetic carnauba, PEG-12 carnauba, myrica wax, rice bran wax, soy wax, ceresin, hydrogenated microcrystalline wax, jojoba wax, microcrystalline wax, lanolin wax, ozokerite, paraffin, synthetic paraffin, cetyl esters, C₂₀-C₄₀ alkyl behenate, cetyl palmitate, stearyl palmitate, lauryl behenate, cetyl octanoate, cetyl ricinoleate, cetyl stearate, decyl oleate, myristyl stearate, lauryl stearate, octyldodecyl stearate, octyldodecyl stearoyl stearate, oleyl arachidate, oleyl stearate, tridecyl stearate, pentaerythrityl tetrabehenate, pentaerythrityl distearate, polyethylene, hydrogenated cottonseed oil, hydrogenated vegetable oil, hydrogenated jojoba oil, hydrogenated palm oil, hydrogenated palm kernel oil, hydrogenated olive oil, polyamides, C₃₀-C₆₀ fatty alcohols, polypropylene, polybutane, zinc stearate, stearyl palmitate, stearyl behenate, shellac wax, glycol montanate, C₂₀-C₄₀ alkyl hydroxystearyl stearate, tribehenin, or combinations thereof. Notably, waxes that have a melting point between about 50° C. to 100° C. may act as setting agents to solidify a formulation. Moreover, plant-based polymer thickeners, mineral thickeners, and wax thickeners may not require neutralization and therefore may be present as un-neutralized thickeners.

Synthetic thickeners may include crosslinked polymers such as, without limitation, alkyl acrylate polymers in which the co-monomers consist of at least one of the following: acrylic acid, sodium acrylate, methacrylic acid, or alkyl acrylate. Polymers consisting purely of acrylic acid are often referred to as carbomers. Thus, for example, single molecules of carbomers can be crosslinked to provide a crosspolymer with an average molecular weight (MW) over 1 billion Daltons (DA). In one example, acrylic acid may be crosslinked with allyl sucrose or allyl pentaerythritol. Similarly, a copolymer of C₁₀-C₃₀ alkyl acrylates and one or more monomers of acrylic acid, methacrylic acid or one of their simple esters may be crosslinked (e.g., with an allyl (2-propenyl) ether of sucrose or an allyl ether of pentaerythritol) to form acrylates/C₁₀-C₃₀ alkyl acrylate crosspolymer. Traditionally, crosslinks of synthetic thickeners are achieved by neutralization agents such as sodium hydroxide, potassium hydroxide, potassium bicarbonate, borax, amino acids, polar organic amines, or combinations thereof. Accordingly, synthetic polymer thickeners may require neutralization and therefore may be present in personal care compositions as neutralized thickeners.

Thickeners may be present from about 0.01 wt. % to about 40.0 wt. %, based on the total weight of the personal care composition. A plant-based, mineral, or synthetic polymer thickener may, for example, be present in an amount of 0.05 wt. %, 0.1 wt. %, 0.5 wt. %, 1.0 wt. %, 2.0 wt. %, 3.0 wt. %, 3.5 wt. %, 4.0 wt. %, including every number and/or fraction therebetween, based on the total weight of the personal care composition. Meanwhile, wax thickeners used as a solidifying agent may be present in an amount up to about 30.0 wt. % such as, for example, 1.0 wt. %, 2.0 wt. %, 5.0 wt. %, 18.0 wt. %, 25.0 wt. %, including every number and/or fraction therebetween, based on the total weight of the composition.

### Carrier(s)

Personal care compositions may include carrier ingredients for use in cosmetic or pharmaceutical compositions to provide various formulation formats such as, without limitation, a gel, a lotion, a cream, a spray, a mist, a stick, a mousse, a foam, an ointment, a milk, a base, a concentrate, a mist, a powder, or combinations thereof. Personal care compositions may, for example, be composed of one or more carriers including, without limitation, water, vegetable oils, esters (e.g., octyl palmitate, isopropyl myristate, isopropyl palmitate, etc.), ethers (e.g., dicapryl ether, dimethyl isosorbide, etc.), C₂-C₄ alcohol (e.g., ethanol, isopropanol, etc.), fatty alcohols (e.g., cetyl alcohol, stearyl alcohol, behenyl alcohol, etc.), paraffins and isoparaffins (e.g., undecane, tridecane, isooctane, isododecane, isohexadecane, etc.), silicone oils (e.g., dimethicones, polysiloxanes, etc.), hydrocarbon oils (e.g., mineral oil, petrolatum, isoeicosane polyisobutene, etc.), polyols (e.g., propylene glycol, glycerin, butylene glycol, pentylene glycol, hexylene glycol, etc.), or combinations thereof.

For example, personal care compositions may be composed of one or more carriers that act as skin-conditioning agents (e.g., emollients, humectants, etc.) for moisturization, etc. Examples of useful skin-conditioning agents include, without limitation, esters (e.g., isopropyl myristate, isopropyl palmitate, 1-decene polymer (hydrogenated), esters of benzoic acid such as lauryl/myristyl benzoate, C₁₂-C₁₅ alkyl benzoate, C₁₆-C₁₇ alkyl benzoate, stearyl benzoate, isostearyl benzoate, behenyl benzoate, ethylhexyl benzoate, butyloctyl benzoate, hexyldecyl benzoate, octyldodecyl benzoate, etc.), polyols (e.g., glycerin, butylene glycol, hexylene glycol, 2,3-butanediol, 1,2-alkane diols such propylene glycol, 1,2-butanediol, pentylene glycol, ethyl hexanediol, caprylyl glycol, decylene glycol, etc.), vegetable and hydrogenated vegetable or natural oils (e.g., nut oil, seed oil, fruit oil, safflower oil, castor oil, coconut oil, cottonseed oil, menhaden oil, palm kernel oil, palm oil, peanut oil, soybean oil, rapeseed oil, linseed oil, rice bran oil, pine oil, sesame oil, sunflower seed oil, partially and fully hydrogenated oils from the foregoing sources. etc.), fatty acids and oils thereof (e.g., lanolin, petrolatum, mineral oil, etc.), triglycerides (caprylic/capric triglyceride, etc.), silicones (cyclomethicone, dimethicone, etc.), salicylates (e.g., butyloctyl salicylate, C₁₂-C₁₅ alkyl salicylate, capryloyl salicylic acid, hexydodecyl salicylate, isocetyl salicylate, isodecyl salicylate, tridecyl salicylate, etc.), or combinations.

Accordingly, in one example, an oil gel may be formulated using mineral oil and a gelling agent (e.g., hydrogenated butylene/ethylene/styrene copolymer, hydrogenated ethylene/propylene/styrene copolymer, etc.) present in an amount of about 0.1 wt. % to about 5.0 wt. %, based on a total weight of the personal care composition. Thickeners, discussed in further detail above, may also be used as gelling agents in hydrous or anhydrous personal care compositions. In another example, alcoholic gels may include one or more lower alcohols or polyols, such as ethanol, propylene glycol or glycerin, and a thickener, such as siliceous earth. Oily-alcoholic gels may also include natural or synthetic oil or wax. Non-aqueous carriers may, however, be used without gelling agents for various applications. For example, mineral oil may be present in an amount of 50.0 wt. % or greater (e.g., >90 wt. %), based on a total weight of a sunscreen composition. Oils may form oil-in-oil or oil-in-silicone or silicone-in-oil emulsions (e.g., castor oil in silicone oil, silicone oil in silicone oil, etc.) to deliver water-insoluble active ingredients (e.g., sunscreen actives, insect repellants, skin care actives, etc.) to a keratinous substrate.

Similarly, paraffins and isoparaffins may be useful non-aqueous carriers. For example, isododecane or isohexadecane may be used. Also useful may be C₄-C₂₀ alkyl ethers of polypropylene glycols, C₁-C₂₀ carboxylic acid esters of polypropylene glycols, di-C₈-C₃₀ alkyl ethers, or combinations thereof. For example, PPG-14 butyl ether, PPG-15 stearyl ether, dioctyl ether, and/or dodecyl octyl, ether may be used. Meanwhile, silicones including those that are non-volatile or volatile and those that are water soluble or water insoluble, or combinations thereof, may include organo-substituted polysiloxanes, which are either linear or cyclic polymers of monomeric silicone/oxygen monomers. Organopolysiloxanes may be selected from a wide variety of silicones spanning a broad range of volatilities and viscosities. For example, organopolysiloxanes may be selected from the group consisting of polyalkylsiloxanes, alkyl substituted dimethicones, dimethiconols, polyalkylaryl siloxanes, or combinations thereof. Thus, silicones may include dimethicone, cetyl dimethicone, cetyl dimethicone copolyol, cyclomethicone, cyclopentasiloxane, dimethicone copolyol, or combinations thereof. Volatile non-aqueous carriers may be preferred in some aspects of embodiments.

Water may also be intentionally added in a personal care composition at an amount greater than 15.0 wt. % such as, for example, 25.0 wt. %, 45.0 wt. %, 55.0 wt. %, 65.0 wt. %, 75.0 wt. %, including every number and/or fraction therebetween, based on the total weight of a hydrous personal care composition. For example, a water-in-oil (W/O) emulsion may have an aqueous dispersed phase stabilized by an emulsifier and disposed in a hydrophobic continuous phase. Water may, however, be intentionally added in an amount of 15.0 wt. % or less, based on a total weight of the personal care composition, to form a substantially anhydrous personal care composition. Moreover, one or more ingredients may have associated with it absorbed and/or adsorbed water that can be carried into the otherwise substantially anhydrous personal care composition. For economic, sourcing, and/or formulation reasons, it may be disadvantageous to process hygroscopic materials to make them completely free of water. Thus, a substantially anhydrous personal care composition may include 15.0 wt. % or less, preferably 10.0 wt. % or less and more preferably 5.0 wt. % or less (e.g., 1.0 wt. %), of water based on a total weight of the personal care composition.

Carriers may be present from about 0.1 wt. % to about 99.0 wt. %, based on the total weight of the personal care composition. A carrier may, for example, be present in an amount of 0.008 wt. %, 0.1 wt. %, 0.5 wt. %, 1.0 wt. %, 2.5 wt. %, 5.0 wt. %, 7.5 wt. %, 10.0 wt. %, 15.0 wt. %, 20.0 wt. %, 25.0 wt. %, 30.0 wt. %, 35.0 wt. %, 40.0 wt. %, 45.0 wt. %, 50.0 wt. %, 55.0 wt. %, 60.0 wt. %, 65.0 wt. %, 70.0 wt. %, 75.0 wt. %, 80.0 wt. %, 85.0 wt. %, 90.0 wt. %, 95.0 wt. %, 99.0 wt. %, including every number and/or fraction therebetween, based on the total weight of the personal care composition. For example, an isoparaffin and/or a silicone may be present in an amount of about 10 wt. % to about 80 wt. %, such as, for example, 8.0 wt. %, 10.0 wt. %, 15.0 wt. %, 20.0 wt. %, 25.0 wt. %, 30.0 wt. %, 35.0 wt. %, 40.0 wt. %, 45.0 wt. %, 50.0 wt. %, 55.0 wt. %, 60.0 wt. %, 65.0 wt. %, 70.0 wt. %, 75.0 wt. %, 80.0 wt. %, 85.0 wt. %, 90.0 wt. %, including every number and/or fraction therebetween, based on a total weight of a personal care composition.

### Surfactant(s)

Embodiments may include surfactants such as anionic, cationic, nonionic, amphoteric, or combinations thereof. Examples of useful anionic surfactants include, without limitation, carboxylates, amino acid derivatives, alkyl sulphates, alkyl ether sulfates, sulphonates, isethionates, taurates, sulfosuccinates, alkyl sulfoacetates, phosphates, alkyl phosphates, or combinations thereof. Examples of useful cationic surfactants include, without limitation, alkylamines, alkylimidazolines, quaternary ammonium compounds (QAC), or combinations thereof. Examples of useful nonionic surfactants include, without limitation, alkanolamides, amine oxides, esterified carboxylic acids, ethoxylated alcohols, poloxamers, or combinations thereof. Examples of useful amphoteric/zwitterionic surfactants include, without limitation, betaines, sultaines, cocamphoacetates, or combinations thereof.

Examples of emulsifiers, which are surfactants that can stabilize emulsions, may include oil-in-water (O/W) emulsifiers, W/O emulsifiers, or combinations thereof. An O/W emulsifier may have a HLB≥7, preferably 7 to 10. Examples of useful O/W emulsifiers include, without limitation, certain sucrose esters (e.g., sucrose stearate, sucrose laurate, etc.), sorbitan esters (e.g., sorbitan laurate, etc.), polyglyceryl esters (e.g., polyglyceryl-3 methylglucose distearate, etc.), ethoxylated fatty ethers and ethoxylated fatty esters having a HLB≥7, or combinations thereof. Examples of useful natural O/W emulsifiers include, without limitation, cetearyl alcohol (and) cetearyl glucoside, glyceryl stearate (and) cetearyl alcohol (and) sodium stearoyl lactylate, cetearyl wheat straw glycosides (and) cetearyl alcohol, cetearyl olivate (and) sorbitan olivate, or combinations thereof. Thus, for example, personal care composition may include O/W emulsifiers such as glyceryl stearate (and) PEG-100 stearate, cetearyl alcohol (and) coco-glucoside, cetearyl alcohol (and) dicetyl phosphate (and) ceteth-10 phosphate, or combinations thereof. In this regard, the term “(and)” is an INCI designation for a mixture of listed components separated by the term.

Meanwhile, a W/O emulsifier may have a hydrophilic-lipophilic balance (HLB) of <7, preferably from 4 to 6. Examples of W/O emulsifiers include, without limitation, certain sucrose esters (e.g., sucrose distearate, etc.), sorbitan esters (e.g., sorbitan oleate, sorbitan dioleate, sorbitan stearate, etc.), polyglyceryl esters (e.g., polyglyceryl-4 isostearate, polyglyceryl dimerate isostearate, etc.), silicones (e.g., a copolymer of a silicone, an alkoxylated derivative of a silicone, or combinations thereof, such as cetyl polyethylene glycol (PEG)/polypropylene glycol (PPG)-10/1 dimethicone, lauryl PEG-8 dimethicone, etc.), fatty acid esters (e.g., glyceryl stearate, etc.), or combinations thereof. Examples of useful natural W/O emulsifiers include cetearyl olivate.

It is, however, understood that other emulsion formats may similarly be used such as oil-in-oil or oil-in-silicone emulsions (e.g., castor oil in silicone oil, etc.) or water-in-silicone emulsions to deliver ingredients (e.g., sunscreen filters, antioxidants, etc.) to a keratinous substrate. Silicone emulsifiers are commercially available (e.g., Shin-Etsu, under the KSG, USG, and KF trade name series), which are compatible with silicones, organic oils, and esters. Silicone emulsifiers may include, for example, polyether-modified alkyl-branched silicone emulsifiers such as cetyl PEG/PPG-10/1 dimethicone and lauryl PEG-8 dimethicone, polyether/alkyl co-modified branched silicone emulsifiers such as lauryl PEG-9 polydimethylsiloxyethyl dimethicone, polyglycerin/alkyl co-modified branched silicone emulsifiers such as lauryl polyglyceryl-3 polydimethyldiloxyethyl dimethicone, polyglycerin-modified branched silicone emulsifiers such as polyglyceryl-3 disiloxane dimethicone, polyether-modified silicone emulsifiers such as PEG/PPG-20/22 butyl ether dimethicone, PEG-10 dimethicone, PEG-11 methyl ether dimethicone, PEG-3 dimethicone, PEG-32 methyl ether dimethicone, emulsifying silicone elastomer gel agents such as dimethicone/polyglycerin-3 crosspolymer, PEG-10/lauryl dimethicone crosspolymer, PEG-15/lauryl dimethicone crosspolymer, PEG-15/lauryl polydimethylsiloxyethyl dimethicone crosspolymer, dimethicone/phenyl vinyl dimethicone crosspolymer, or combinations thereof. Thus, for example, personal care compositions may include W/O emulsifiers such as lauryl PEG-8 dimethicone, cetyl PEG/PPG-10/1 dimethicone, lauryl polyglyceryl-3 polydimethylsiloxyethyl dimethicone, or combinations thereof.

Surfactants may be present from about 0.1 wt. % to about 20.0 wt. %, based on the total weight of the personal care composition. One or more surfactants may, for example, be present in an amount of 0.008 wt. %, 0.1 wt. %, 0.5 wt. %, 1.0 wt. %, 2.0 wt. %, 3.0 wt. %, 4.0 wt. %, 5.0 wt. %, 6.0 wt. %, 7.0 wt. %, 8.0 wt. %, 9.0 wt. %, 10.0 wt. %, 11.0 wt. %, 12.0, 13.0 wt. %, 14.0 wt. %, 15.0 wt. %, 16.0 wt. %, 17.0 wt. %, 18.0 wt. %, 19.0 wt. %, 20.0 wt. %, 21.0 wt. %, 22.0 wt. %, including every number and/or fraction therebetween, based on the total weight of the personal care composition.

### Film Former(s)

Personal care compositions may include film former polymers capable of providing a substantially homogenous and/or structured hydro-lipidic film on skin. Generally, a film former polymer may be synthesized from two or more different monomers to form copolymers and/or may from individual polymer chains that are connected by bridging molecules or crosslinking agents to form crosslinked copolymers known as cross polymers. For example, film former polymers may include, without limitation, monomers, copolymers, crosspolymers, and/or terpolymers of an organo-silicone hybrid, (meth)acrylate, abietic acid derivative, olefin, silicone, resin, vinyl acetate (VA), vinyl pyrrolidone (VP), maleate, alkyl ester, long chain or short chain carboxylic acid, or combinations thereof. As discussed above, certain crosspolymers may primarily or exclusively provide gelling, rheology modifier, binder, or absorbent functionality owing to their structure and/or activation. Accordingly, while such crosspolymers may be used for rheology purposes, particularly useful polymers to form films may have a MW of about 1,500,000 DA or less.

Film former polymers may include crotonic acid/vinyl C₈-C₁₂ isoalkyl esters/VA/bis-vinyldimethicone crosspolymer, acrylate/dimethicone copolymer, VA/butyl maleate/isobornyl acrylate copolymer, acrylates/C₁₂-C₂₂ alkyl methacrylate copolymer, acrylates/octylacrylamide copolymer, methyl dihydroabietate, trimethylsiloxysilicate, C₂₄-C₂₈ alkyldimethylsiloxy trimethylsiloxysilicate, polymethylsilsesquioxane/trimethylsiloxysilicate, polymethylsilsesquioxane, perfluorononyl dimethicone, trifluoropropyldimethylsiloxy/trimethylsiloxy silsesquioxane, PPG-17/isophorone diisocyanate/dimethylol propionic acid copolymer, polyurethane (e.g., 32, 34, 35, 48, etc.), hydrolyzed wheat protein/PVP crosspolymers, maltodextrin/VP copolymer, butylated PVP copolymer, VP/polycarbamylpolyglycol ester, VP/dimethylaminoethylmethacrylate/polycarbamyl polyglycol ester, VP/Dimethiconyl-acrylate/polycarbamyl polyglycol ester, VP/eicosene copolymer, adipic acid/diglycol crosspolymer, trimethylpentanediol/adipic acid/glycerin crosspolymer, polyester-7 (and) neopentyl glycol diheptanoate, polyester-10 (and) propylene glycol dibenzoate, hydrogenated polycyclopentadiene (and) isododecane, stearyl/octyldodecyl citrate crosspolymer, hydroxyethyl acrylate/sodium acryloyldimethyl taurate copolymer, or combinations thereof.

A film former polymer may be dispersed in a premix. For example, a non-aqueous dispersant may include an alcohol, an ether, a fatty acid, a silicone fluid, an ester, a hydrocarbon, a fluorinated hydrocarbon, or combinations thereof. A film former polymer may be present in an aqueous solvent, for example dispersed in water. In one example, a (meth)acrylate film former polymer may be present in a % solids content of about 20 wt. % to 100 wt. %, for example 30 wt. % to 40 wt. %, based on the total weight of the dispersion.

A film former polymer may be present in an amount of about 0.1 wt. % to about 5.0 wt. %, based on the total weight of the sunscreen composition. For example, one or more film former polymers may be present in an amount of 0.08 wt. %, 0.5 wt. %, 1.0 wt. %, 2.0 wt. %, 2.5 wt. %, 3.5 wt. %, 5.0 wt. %, 6.0 wt. %, including every number and/or fraction therebetween, based on the total weight of the sunscreen composition.

### Sunscreen Filter(s)

A personal care composition may include a sunscreen filter capable of substantially absorbing, scattering, and/or deflecting UV radiation on a keratinous substrate. Preferably, the sun protection factor (SPF) of a sunscreen composition is >8 and the UVA protection factor (UVAPF) is at least ⅓ the SPF. Thus, for example, physical or mineral sunscreen filters include metal oxides (e.g., titanium dioxide, zinc oxide, combinations thereof, etc.) that may be used in effective amounts to provide an SPF of ≥8 or more (in contrast to other uses as colorants, etc.). Such filters may have various morphologies such as crystalline form (e.g., rutile, etc.), size (e.g., nano, non-nano, etc.), shape (e.g., amorphous, spherical, etc.), and functionalization (e.g., coatings, etc.) that allow for the effective use as sunscreen filters.

Organic sunscreen filters include one or more of: p-aminobenzoic acids such as p-aminobenzoic acid (CAS #: 150-13-0), p-aminobenzoic acid, monoglyceryl ester (CAS #: 136-44-7), p-aminobenzoic acid, octyl dimethyl ester (padimate-0, CAS #: 21245-02-3), p-aminobenzoic acid, ethyl dihydroxypropyl (roxadimate, CAS #: 58882-17-0), p-aminobenzoic acid, 2,4-dihydroxy-N-(3-hydroxypropyl)-3,3-dimethylbutanamide ester, p-aminobenzoic acid, PEG-25 (CAS #: 116242-27-4); anthranilates such as menthyl anthranilate (CAS #: 134-09-8); benzophenones such as benzophenone (CAS #: 119-61-9), 2,4-dihydroxybenzophenone (benzophenone-1, CAS #: 131-56-6), 2,2′,4,4′-tetrahydroxybenzophenone (benzophenone-2, CAS #: 131-55-5), 2-hydroxy-4-methoxybenzophenone (benzophenone-3, oxybenzone, CAS #: 131-57-7), 2-hydroxy-4-methoxybenzophenone-5-sulfonic acid (benzophenone-4, sulisobenzone, CAS #: 4065-45-6), 2-hydroxy-4-methoxybenzophenone-5-sulfonic acid, monosodium salt (benzophenone-5, CAS #: 6628-37-1), 2,2′-dihydroxy-4,4′-dimethoxybenzophenone (benzophenone-6, CAS #: 131-54-4), 5-chloro-2-hydroxybenzophenone (benzophenone-7, CAS #: 85-19-8), 2,2′-dihydroxy-4-methoxybenzophenone (benzophenone-8, dioxybenzone, CAS #: 131-53-3), 2,2′-dihydroxy-4,4′-dimethoxybenzophenone-5,5′-disulfonic acid sodium salt (benzophenone-9, CAS #: 76656-36-5), 2-hydroxy-4-methoxy-4′-methyl-benzophenone (benzophenone-10, Mexenone, CAS #: 1641-17-4), bis(2,4-ihydroxyphenyl)methanone (benzophenone-11, CAS #: 1341-54-4), 2-hydroxy-4-(octyloxy)benzophenone (benzophenone-12, octabenzone, CAS #: 1843-05-6), 2,2′-dihydroxy-4-methoxybenzophenone (dioxybenzone, CAS #: 131-53-3), 2-hydroxy-4-methoxybenzophenone (oxybenzone, CAS #: 131-57-7), 2-hydroxy-4-methoxybenzophenone-5-sulphonic acid (sulisobenzone, CAS #: 4065-45-6), hexyl 2-(4-(diethylamino)-2-hydroxybenzoyl)benzoate (CAS #: 302776-68-7); camphors such as benzylidene camphor sulfonic acid (CAS #: 56039-58-8), 3-benzylidene camphor (CAS #: 15087-24-8), polyacrylamidomethyl benzylidene camphor (CAS #: 113783-61-2), terephthalylidene dicamphor sulfonic acid (CAS #: 90457-82-2), 3-(4-methylbenzyliden) camphor (CAS #: 36861-47-9), camphor benzalkonium methosulfate (CAS #: 52793-97-2), bornelone (CAS #: 2226-11-1); cinnamates such as ethyl cinnamate (CAS #: 103-36-6), 2-ethoxyethyl-p-methoxycinnamate (cinoxate, CAS #: 104-28-9), isoamyl p-methoxycinnamate (CAS #: 71617-10-2), diisopropyl methyl cinnamate (CAS #: 32580-71-5), 2-ethylhexyl alpha-cyano-beta-phenylcinnamate (octocrylene, CAS #: 6197-30-4), diethanolamine methoxycinnamate (CAS #: 56265-46-4), isopropyl methoxycinnamate (CAS #: 5466-76-2), isoamyl p-methoxycinnamate (CAS #: 71617-10-2), glyceryl octanoate dimethoxycinnamate, ethyl diisopropylcinnamate (CAS #: 32580-72-6), ethyl methoxycinnamate (CAS #: 99880-64-5), octyl methoxycinnamate (octinoxate, CAS #: 5466-77-3); dibenzoylmethanes such as dibenzoylmethane (CAS #: 120-46-7), isopropyl dibenzoylmethane (eusolex, CAS #: 63250-25-9), 4-tert-Butyl-4′-methoxy-dibenzoylmethane (avobenzone, CAS #: 70356-09-1); salicylates such as 3,3,5-trimethylcyclohexyl salicylate (homosalate, CAS #: 118-56-9), benzyl salicylate (CAS #: 118-58-1), octyl salicylate (octisalate, CAS #: 118-60-5), 2-hydroxyethyl salicylate (CAS #: 87-28-5), methyl salicylate (CAS #: 89-46-3), isopropylbenzyl salicylate (CAS #: 94134-93-7); imidazoles such as phenylbenzimidazole, phenylbenzimidazol-5-sulfonic acid (CAS #: 27503-81-7), phenylbenzimidazole tea sulfonate (CAS #: 73705-00-7), urocanic acid [4-imidazoleacrylic acid, CAS #: 104-98-3], ethyl urocanate (CAS #: 27538-35-8), bisymidazylate (CAS #: 180898-37-7), sodium phenylbenzimidazole sulfonate (CAS #: 5997-53-5); triazines such as bis(ethylhexyloxyphenol methoxyphenol) triazine (bemotrizinol, CAS #: 187393-00-6); triazoles such as 2-(2-hydroxy-5-methyl-phenyl)benzotriazole (drometrizole, CAS #: 2440-22-4); triazones such as diethylhexyl butamido triazone (iscotrizinol, CAS #: 154702-15-5), ethylhexyl triazone (CAS #: 88122-99-0); petrolatum (CAS #: 8009-03-8), acrylates such as diurethane dimethacrylate (CAS #: 103597-45-1), ethyl 2-cyano-3,3-diphenylacrylate (etocrilene, CAS #: 5232-99-5); siloxanes such as drometrizole trisiloxane (CAS #: 155633-54-8); or combinations thereof.

A sunscreen filter that protects against UVA radiation may include avobenzone, diethylamino hydroxybenzoyl hexyl benzoate, terephthalylidene dicamphor sulfonic acid, bis-disulizole disodium, disodium phenyl dibenzimidazole tetrasulfonate, bis-diethylamino hydroxybenzoyl benzoate, bis-benzoxazolylphenyl ethylhexylamino triazine, or combinations thereof. A sunscreen filter that protects against UVB radiation may include octocrylene, octinoxate, octisalate, homosalate, ensulizole, ethylhexyl triazone, enzacamene, amiloxate, iscotrizinol, benzylidene malonate polysiloxane, padimate-O, trolamine salicylate, cinoxate, PABA, or combinations thereof. A sunscreen filter that protects against UVA and UVB radiation may include oxybenzone, meradimate, titanium dioxide, zinc oxide, bis-octrizole, bis-ethylhexyloxyphenol methoxyphenyl triazine, drometrizole trisiloxane, sulisobenzone, dioxybenzone, or combinations thereof. A sunscreen filter that protects against damaging radiation within other regions in the electromagnetic spectrum, such as blue light (380 nm to 500 nm) or IR (700 nm-1 mm), may include, without limitation, titanium dioxide, zinc oxide, combinations thereof.

A sunscreen filter may, for example, be present in an amount of about 1.0 wt. % to 40.0 wt. %, based on the total weight of the sunscreen composition. For example, one or more sunscreen filters may be present in an amount of 0.8 wt. %, 1.0 wt. %, 5.0 wt. %, 10.0 wt. %, 15.0 wt. %, 20.0 wt. %, 30.0 wt. %, 41.0 wt. %, including every number and/or fraction therebetween, based on the total weight of the sunscreen composition.

### Additional Ingredient(s)

Embodiments may include other ingredients such as, without limitation, anti-aging actives, insect repellants, amino acids, colorants, pigments, dyes, opacifiers, pH adjusters, fragrances, fillers, preservatives, preservative boosters, chelating agents, stabilizers, sphingolipids, minerals, keratolytics, hormonal compounds, analgesics, anti-allergenic agents, H1 or H2 antihistamines, anti-inflammatory agents, anti-irritants, anti-neoplastics, immune system boosting agents, immune system suppressing agents, anti-acne agents, anesthetics, antiseptics, skin cooling compounds, skin pollution protectants, skin penetration enhancers, exfoliants, staining agents, depigmenting agents, hypopigmenting agents, stabilizers, additional extracts (fruit, flower, plant, vegetable), additional vitamins, alpha and beta hydroxy acids, retinol and its derivatives, odor neutralizers, elastomers, propellants, or combinations thereof.

Examples of skin care actives that provide skin benefits may include, for example, anti-acne ingredients, anti-aging ingredients, exfoliation ingredients, or combinations thereof. Anti-acne actives may include, without limitation, benzoyl peroxide, salicylic acid, retinoids (e.g., adapalene, tretinoin, etc.), azelaic acid, antibiotics (clindamycin, erythromycin, sulfacetamide, etc.), sulfur ingredients, or combinations thereof. Anti-aging ingredients may include retinol, bakuchiol, hyaluronic acid, or combinations thereof. For example, a skin care composition may include bakuchiol and/or hyaluronic acid to improve skin tone, improve skin elasticity, smooth fine lines, smooth wrinkles, or combinations thereof. Bakuchiol may preferably be present with an oil-based skin conditioning agent selected from hemp oil, bisabolol, adansonia digitata seed oil, or combinations thereof.

Exfoliation particles may include any material suitable to exfoliate the skin such as, without limitation, fruit seeds, fruit stones, nut shells, ground or fibrous plant material, polymers (e.g., natural, synthetic, etc.), mineral composites, or combinations thereof. Chemical exfoliants may additionally or alternatively be used. For example, allantoin, also known as aluminum dihydroxy allantoinate, may have keratolytic properties to assist in desquamation, the shedding of the outermost membrane or layer of tissue from the inside out. Additionally or alternatively, allantoin may soothe injured skin and promote wound healing, crease the water contents of cells, promote cell proliferation and supports overall remodeling, smooth skin, stimulate fibroblasts (cells responsible for producing collagen), boost extracellular matrix synthesis, or combinations thereof.

Meanwhile, odor neutralizers or absorbers may include perfumes, mineral powders, polymers (e.g., polymers in salt form), or combinations thereof. Perfumes may include, without limitation, naturally derived fragrances (extracts, essential oils, etc.), synthetically derived fragrances (small molecules), or combinations thereof. Mineral powder may include, without limitation, silica, silicate, bentonite, hectorite, smectite, kaolin, saponite, carbonate, or combinations. Polymers may include, without limitation, n-soya-n-ethyl morpholinium ethosulfate, cyclodextrins, or combinations thereof. Thus, in one example, metal oxide silicates (e.g., calcium silicate) may act as odor absorbents and neutralizers to absorb and neutralize body malodors. In a further example, cyclodextrins may be used to entrap active ingredients and thereby control the release of polyols, anti-microbials, zinc salts, polymers, bicarbonate salts, chelating agents, zeolites and activated carbon. Similarly, silicates, silicas and carbonates may absorb moisture which may indirectly reduce malodor formation by eliminating cutaneous flora that cause the odor.

Elastomers may include natural or synthetic materials, including polymers, that substantially resume their original shape when a deforming force is removed. Silicone elastomers, for example, are a broad class of cross-linked dimethicone polymers that form three-dimensional polymeric structures. Interstitial pore volume, defined by the cross-link density of the silicones, can be swollen (filled) with a solvent or even a relatively small active material. Silicone elastomers may refer to silicone crosspolymers, dimethicone crosspolymers, dimethicone/vinyl dimethicone crosspolymers, Polysilicone-11, and so on. Thus, for example, the elastomer may be selected from dimethicone/dimethicone crosspolymers, dimethicone/vinyldimethicone crosspolymers, silicone crosspolymers, silicone, or combinations thereof.

Silicone elastomers are commercially available (e.g., Shin-Etsu, under the KSG, USG, and KF trade name series). Thus, for example, silicone elastomers may include dimethicone/phenyl vinyl dimethicone crosspolymer, dimethicone/polyglycerin-3 crosspolymer, lauryl polydimethylsiloxyethyl dimethicone/bis-vinyldimethicone crosspolymer, PEG-10/lauryl dimethicone crosspolymer, PEG-15/lauryl dimethicone crosspolymer, PEG-15/lauryl polydimethylsiloxyethyl dimethicone crosspolymer, dimethicone/phenyl vinyl dimethicone crosspolymer, diphenylsilocy phenyl thimethicone, polyglyceryl-3/lauryl poilydimethylsiloxyethyl dimethicone crosspolymer, vinyl dimethicone/lauryl dimethicone crosspolymer, or combinations thereof. Notably, as described above, one or more silicone elastomers may operatively function as an emulsifying silicone elastomer gel agent in various formulation formats.

Silicone elastomers are available as loose powders, as swollen mixtures in cosmetic solvents, and as dispersions in water. The basic polymers can be further compounded into anhydrous and aqueous formula bases. Creams and lotions may, for example, contain a substantial amount of silicone elastomer (e.g., up to about 20 wt. %, up to about 10 wt. %, etc.). In one example, an invert emulsion includes water (dispersed/internal phase) suspended or dispersed throughout a continuous/external phase including a volatile silicone carrier, includes a W/O emulsifier, and includes a silicone elastomer present in an amount from about 0.1 wt. % to about 10.0 wt. %, based on the total weight of the sunscreen composition.

The amount of additional ingredients may be present from about 0.001 wt. % to about 90.0 wt. %, based on the total weight of the sunscreen composition. One or more additional ingredients may, for example, be present in an amount of 0.0008 wt. %, 0.01 wt. %, 0.08 wt. %, 1.0 wt. %, 2.0 wt. %, 5.0 wt. %, 10.0 wt. %, 20.0 wt. %, 30.0 wt. %, 40.0 wt. %, 50.0 wt. %, 60.0 wt. %, 70.0 wt. %, 80.0 wt. %, 91.8 wt. %, including every number and/or fraction therebetween, based on the total weight of the sunscreen composition. A total amount of all additional ingredients may be sufficient to bring the total amount of the sunscreen composition up to 100 wt. %.

### Formulation and Product Format(s)

Personal care compositions may include one or more formulation formats such as, without limitation, an emulsion (O/W, W/O, multiple emulsions, silicone emulsions, etc.), a gel, a lotion, a cream, a spray, a mist, a stick, a mousse, a foam, an ointment, a milk, a base, a concentrate, a mist, a powder, or combinations thereof. Such formats may initially or finally be substantially aqueous or substantially anhydrous. For example, a substantially anhydrous base may be used in an aqueous emulsion to form a lotion or a cream. Similarly, ingredients and use levels to make such formats may be readily adjusted and mixed in a single phase or in multiple phases to provide a desired commercial personal care product. Thus, for example, water content, alcohol content, wax content, emulsifiers, etc., may be included, omitted, adjusted, or mixed in a single phase or in multiple phases to provide commercial products that account for cost, aesthetics, and/or product performance objectives.

Moreover, personal care compositions can be filled into any number of bottles, tottles, tubes, packets, etc., having a variety of closing structures, pumps, sprays (e.g., mists, aerosols, bag on valve), amongst others, or combinations thereof. A bag-on-valve system may, for example, be used to deliver the sunscreen composition using compressed gas such as air, nitrogen, or carbon dioxide. It is, however, understood that personal care compositions may be used in any product format including any similar to formats discussed herein.

For example, a personal care composition may be formulated as a concentrate which, when combined with a propellant, forms a continuous aerosol spray commercial product. Propellants may include, for example, isobutene, butane, propane, dimethyl ether, methyl ether, tetrafluoroethane, 1,1-difluoroethane, or combinations thereof. Propellants generally exist as equilibrium of vapor and liquid and can be either dissolved in or miscible with the composition. Ratios of propellant to the concentrate can be adjusted to account for cost, aesthetics, and product performance objectives. In one embodiment, the concentrate and the propellant are added to an aerosol container in a ratio of about 90:10 to about 60:40, for example about 75:25, about 70:30, and so on. The aerosol propellant may, for example, be present in an amount of about 10 wt. % to about 60 wt. %, based on a total weight of the personal care composition (e.g., final composition).

### Example(s)

Examples were prepared using known methods. Unless otherwise noted, formulations were mixed in phases with Camellia sinensis (green tea) extract added at the end, when all phases and ingredients were mixed (e.g. post-emulsification), under relatively low temperature conditions (e.g., <40° C.). When neutralized thickeners were used, Camellia sinensis (green tea) extract was added after neutralization. Antioxidants used were added in a crystalline dosage form, aqueous dosage form, or non-aqueous dosage form. When added in a premix dispersion, use levels of Camellia sinensis (green tea) extract refers to the level of the extract itself and not to the level of the dispersion used to obtain the use levels.

Electron Spin Resonance (ESR) Spectrophotometric assays were implemented to measure antioxidant capacity in absorbance units (AU) and transformed to a qualitative scale of antioxidant power (AP) as follows: 1-10 (no antioxidant performance); 11-100 (moderate antioxidant performance); 101-500 (high antioxidant performance); 501-4000 (very high antioxidant performance).

In vitro data was obtained using a Labsphere Ultraviolet Transmittance Analyzer (Model UV-2000 available from the Solar Light Company, Philadelphia, Pennsylvania). The readings may be recorded by the analyzer and the calculation of the SPF values can be based on the following equation:

$$SPF = \frac{{\int }_{\text{?}}^{\text{?}} E \text{?} \cdot {S}_{λ} \cdot d λ}{{\int }_{\text{?}}^{\text{?}} {E}_{λ} \cdot {S}_{λ} \cdot {T}_{λ} \cdot d λ}$$

$$\text{?} \text{indicates text missing or illegible when filed}$$

with built-in software: UV-2000 application Version 1.1.0.0, wherein E_{λ} is the Commission Internationale de l'Eclairage (CIE) erythemal spectral effectiveness, S_{λ} is designated as the solar spectral irradiance, and T_{λ} is the spectral transmittance of the sample as measured on the UV-2000.

### Example O/W Formulations

TABLE I
Example Lotions

| Component (INCI) | Function | Approx. wt. % |
|---|---|---|
| Example Main Ingredients | | |
| Water | Continuous Phase | 50-90 |
| Cetearyl Alcohol (and) Coco-Glucoside, | Emulsifier | 0.5-5.0 |
| Cetearyl Alcohol (and) Dicetyl Phosphate (and) | (HLB ≥ 7) | |
| Ceteth-10 Phosphate, Glyceryl Stearate (and) | | |
| PEG-100 Stearate, Cetyl Alcohol, Polyglyceryl-3 | | |
| Methylglucose Distearate | | |
| Xanthan Gum | Un-neutralized | 0.1-1.0 |
| | Polymer Thickener | |
| Camellia sinensis (green tea) extract | Antioxidant | 0.005-0.2 |
| | (aqueous dispersion) | |
| Additional Ingredients | | |
| Disodium EDTA, Citric Acid, Aminomethyl | Chelating agent, pH | Q.S. 100 |
| Propanol, Sodium Polyphosphate, | Adjuster, preservative | |
| Phenoxyethanol, Chlorphenesin, Octocrylene, | system, Sunscreen, | |
| Homosalate, Ethylhexyl Salicylate, Butyl | Solvent, Skin | |
| Methoxydibenzoylmethane, Zinc Oxide, | Conditioner, | |
| Butyloctyl Salicylate, C₁₂-C₁₅ Alkyl Benzoate, | Additional | |
| Sodium Hyaluronate, Propylheptyl Caprylate, | Antioxidant, Film | |
| Benzyl Alcohol, Caprylyl Glycol, Glycerin, | Former, Dispersant, | |
| Panthenol, Seal Salt, Isohexadecane, | Fragrance | |
| Diisopropyl Adipate, Butylene Glycol, | | |
| Hydroxyethyl Acrylate/Sodium | | |
| Acryloyldimethyl Taurate Copolymer, Oil | | |
| (soybean, grape seed, candlenut seed), Butter | | |
| (cocoa seed, jojoba seed), vitamin E derivative | | |
| (Tocopheryl Acetate), Daucus Carota Sativa | | |
| (Carrot) Root Extract, vitamin C derivative | | |
| (Sodium Ascorbyl Phosphate), Acrylates/C₁₂-C₂₂ | | |
| Alkyl Methacrylate Copolymer (and) Water, | | |
| VP/Eicosene Copolymer, Polyhydroxystearic | | |
| Acid, Alcohol, Aloe, Extract (flower), Fragrance | | |

Personal care compositions were formulated with the ingredients at the levels shown in Table 1. Example 1 (AP=40) included a mixture of glyceryl stearate and PEG-100 stearate together with cetyl alcohol, xanthan gum without carbomer or acrylates/C₁₀₋₃₀ alkyl acrylate crosspolymer, and Camellia sinensis (green tea) extract in an amount from 0.1 wt. % to 0.2 wt. %, based on the total weight of the composition. Camellia sinensis (green tea) extract added was in aqueous dosage form (e.g., with water, a diol, and additives). Additional ingredients included disodium EDTA, phenoxyethanol, chlorphenesin, octocrylene, homosalate, ethylhexyl salicylate, butyl methoxydibenzoylmethane, zinc oxide, glycerin, VP/Eicosene copolymer, sea salt, isohexadecane, polyhydroxystearic acid, silica, alcohol, aloe, oil (soybean), butters (cocoa seed, jojoba seed). Organic and mineral sunscreen filters were present in effective amounts for an SPF of ≥50. Example 2, which was similar to Example 1 except primarily with sunscreen filters present in effective amounts for an SPF of ≥30, similarly exhibited moderate antioxidant performance (AP=37).

Example 3 (AP=103) included polyglyceryl-3 methylglucose distearate and cetyl alcohol, xanthan gum without carbomer or acrylates/C₁₀₋₃₀ alkyl acrylate crosspolymer, and Camellia sinensis (green tea) extract in an amount from 0.1 wt. % to 0.2 wt. %, based on the total weight of the composition. Camellia sinensis (green tea) extract added was in aqueous dosage form (e.g., water, a diol, and additives). Additional ingredients included citric acid, sodium polyphosphate, phenoxyethanol, chlorphenesin, benzyl alcohol, panthenol, tocopheryl acetate, Daucus Carota Sativa (Carrot) root extract, sodium ascorbyl phosphate, glycerin, cetyl alcohol, propylheptyl caprylate, diisopropyl adipate, oil (soybean, candlenut seed), and butter (shea). No sunscreen filters were present in Example 3.

Example 4 (AP=154) included a mixture of cetearyl alcohol and coco-glucoside together with a mixture of cetearyl alcohol, dicetyl phosphate, and ceteth-10 phosphate, xanthan gum without carbomer or acrylates/C₁₀-C₃₀alkyl acrylate crosspolymer, and Camellia sinensis (green tea) extract present in an amount from 0.1 wt. % to 0.2 wt. %, based on the total weight of the composition. Camellia sinensis (green tea) extract added was in aqueous dosage form (e.g., water, a diol, and additives). Additional ingredients included disodium EDTA, aminomethyl propanol, phenoxyethanol, chlorphenesin, octocrylene, homosalate, ethylhexyl salicylate, butyl methoxydibenzoylmethane, butyloctyl salicylate, caprylyl glycol, diisopropyl adipate, butylene glycol, hydroxyethyl acrylate/sodium acryloyldimethyl taurate copolymer (and) isohexadecane (and) polysorbate 60 (and) water, tocopheryl acetate, Daucus Carota Sativa (Carrot) root extract, sodium ascorbyl phosphate, acrylates/C₁₂-C₂₂ alkyl methacrylate copolymer (and) water, oils (soybean, grape seed), aloe, extract (flower) and fragrance. Only organic filters were present in effective amounts for an SPF of ≥50. Additional examples were formulated similar to Example 4, except that ingredients added to those listed in Example 4 included C₁₂-C₁₅ alkyl benzoate, sodium hyaluronate, benzyl alcohol, and panthenol, and that certain examples excluded fragrance.

Accordingly, Camellia sinensis (green tea) extract in an amount from 0.005 wt. % to 0.2 wt. %, initially dispersed in an aqueous solvent (e.g., water, a diol, and other additives), surprisingly enhances antioxidant power and minimizes unnecessary waste. For example, Camellia sinensis (green tea) extract in relatively low amounts from 0.1 wt. % to 0.2 wt. %, based on the total weight of the composition, may provide moderate to high or very high antioxidant performance in O/W emulsions, e.g., when used with a thickener that is present in un-neutralized form (e.g., in the absence of certain or any neutralized thickener) and/or when the extract is added at the end of the manufacturing process (post-emulsification) under relatively low temperature conditions. Moreover, there may be larger antioxidant performance when certain sunscreen filters are omitted (e.g., Examples 1-2) although moderate antioxidant performance can be achieved at relatively low use levels in the presence of mineral and organic sunscreen filters (e.g., Example 3). Also, despite having the same levels of Camellia sinensis (green tea) extract or Daucus Carota Sativa (Carrot) root extract as Examples 1-2 and far less tocopheryl acetate and sodium ascorbyl phosphate than Camellia sinensis (green tea) extract, a marked improvement in antioxidant performance may be achieved when only organic sunscreen filters are present in O/W emulsions (Example 4).

TABLE II
Example Lotions and Butters

| Component (INCI) | Function | Approx. wt. % |
|---|---|---|
| Example Main Ingredients | | |
| Water | Continuous Phase | 50-90 |
| Cetearyl Alcohol (and) Coco-Glucoside, | Emulsifier | 0.5-5.0 |
| Cetearyl Alcohol (and) Dicetyl Phosphate (and) | (HLB ≥ 7) | |
| Ceteth-10 Phosphate, Cetyl Alcohol | | |
| Carbomer, Acrylates/C₁₀₋₃₀ alkyl acrylate | Neutralized Polymer | 0.1-1.0 |
| crosspolymer | Thickener | |
| Xanthan Gum | Un-neutralized | 0.1-1.0 |
| | Polymer Thickener | |
| Camellia sinensis (green tea) extract | Antioxidant | 0.04-0.2 |
| | (aqueous dispersion) | |
| Additional Ingredients | | |
| Disodium EDTA, Sodium Hydroxide, | Chelating agent, | Q.S. 100 |
| Phenoxyethanol, Chlorphenesin, Octocrylene, | Neutralizing agent, | |
| Homosalate, Ethylhexyl Salicylate, Butyl | preservative system, | |
| Methoxydibenzoylmethane, Benzyl Alcohol, | Sunscreen, Solvent, | |
| Caprylyl Glycol, Glycerin, Isopropyl Myristate, | Skin Conditioner, | |
| Glyceryl Stearate, Cetearyl Alcohol, Panthenol, | Additional | |
| Diisopropyl Adipate, Oil (argan, coconut, | Antioxidant, Film | |
| avocado, soybean), Butter (mango, shea, | Former, Colorant | |
| cocoa), Tocopheryl Acetate, Daucus Carota | | |
| Sativa (Carrot) Root Extract, Sodium Ascorbyl | | |
| Phosphate, Acrylates/C₁₂-C₂₂ Alkyl | | |
| Methacrylate Copolymer (and) Water, extracts | | |
| (cocoa), mica (and) titanium dioxide, Aloe, | | |
| vitamin C derivative (ascorbyl isostearate, | | |
| ascorbyl glucoside), vitamin B derivative | | |
| (Niacinamide), Fragrance. | | |

Personal care compositions were formulated with the ingredients at the levels shown in Table 2. Example 5 (AP=128) included a mixture of cetearyl alcohol and coco-glucoside together with a mixture of cetearyl alcohol, dicetyl phosphate, and ceteth-10 phosphate, xanthan gum and acrylates/C₁₀-C₃₀ alkyl acrylate crosspolymer, and Camellia sinensis (green tea) extract present in an amount from 0.1 wt. % to 0.2 wt. %, based on the total weight of the composition. Camellia sinensis (green tea) extract added was in aqueous dosage form (e.g., water, a diol, and additives). Additional ingredients included disodium EDTA, sodium hydroxide, phenoxyethanol, chlorphenesin, octocrylene, homosalate, ethylhexyl salicylate, butyl methoxydibenzoylmethane, benzyl alcohol, glycerin, panthenol (and) water, diisopropyl adipate, tocopheryl acetate, Daucus Carota Sativa (Carrot) root extract, sodium ascorbyl phosphate, acrylates/C₁₂-C₂₂ alkyl methacrylate copolymer (and) water, oils (argan, soybean), butters (mango, shea), extracts (cocoa), and mica (and) titanium dioxide. Only organic filters were present in effective amounts for an SPF of ≥50.

Example 5 was compared to a similar formulation having 0.5 wt. % to <0.1 wt. % Camellia sinensis (green tea) extract (Comparative Example 1), which exhibited markedly lower antioxidant performance (AP=11). This was surprising, since Example 5 had over a ten-fold increase in antioxidant performance with only about 50% more extract (notably, at relatively low use levels). Two O/W formulations (Comparative Examples 2 and 3), having the same level of Camellia sinensis (green tea) extract as Comparable Example 1 with extract added after neutralization, notably exhibited even lower antioxidant power (AP=8).

Example 6 (AP=118) included a mixture of cetearyl alcohol, dicetyl phosphate, and ceteth-10 phosphate together with cetyl alcohol, carbomer without xanthan gum, and Camellia sinensis (green tea) extract in an amount from 0.1 wt. % to 0.2 wt. %, based on the total weight of the composition. Camellia sinensis (green tea) extract added was in aqueous dosage form (e.g., water, a diol, and additives). Additional ingredients included disodium EDTA, sodium hydroxide, phenoxyethanol, chlorphenesin, benzyl alcohol, caprylyl glycol, glycerin, isopropyl myristate, glyceryl stearate, cetearyl alcohol, panthenol, diisopropyl adipate, tocopheryl acetate, Daucus Carota Sativa (Carrot) root extract, oils (coconut, avocado, soybean), butters (shea, cocoa, mango), aloe, vitamin C derivative (ascorbyl isostearate), and fragrance. No sunscreen filters were present in Example 6.

Examples 7-8 (AP=40) included a mixture of cetearyl alcohol and coco-glucoside together with a mixture of cetearyl alcohol, dicetyl phosphate, and ceteth-10 phosphate, carbomer and xanthan gum, and Camellia sinensis (green tea) extract in an amount from 0.040 wt. % to <0.1 wt. %, based on the total weight of the composition. Camellia sinensis (green tea) extract added was in aqueous dosage form (e.g., water, a diol, and additives). Additional ingredients included disodium EDTA, sodium hydroxide, phenoxyethanol, chlorphenesin, benzyl alcohol, glycerin, diisopropyl adipate, tocopheryl acetate, acrylates/C₁₂-C₂₂ alkyl methacrylate copolymer (and) water, oil (coconut), vitamin C derivative (ascorbyl glucoside), and vitamin B derivative (niacinamide). Only organic sunscreen filters were present in effective amounts for an SPF of ≥50. Example 9, which was similar to Examples 7-8 except primarily that sunscreen filters were in effective amounts for an SPF of ≥30, similarly exhibited moderate antioxidant performance.

Comparative Example 3, which exhibited no antioxidant performance (AP=9), was similar to Example 7 except primarily that no Camellia sinensis (green tea) extract was present. Surprisingly, when Camellia sinensis (green tea) extract was present with the vitamin C derivative, e.g., in about a 1:10 ratio, over four-fold increase in antioxidant performance was observed in Example 7 (AP=40) even though the use level of the vitamin C derivative did not change. Example 10, which was similar to Example 7 except primarily that Camellia sinensis (green tea) extract was doubled and therefore was present with the vitamin C derivative in a 1:5 ratio, exhibited high antioxidant performance (AP=161). In this regard, over a ten-fold increase in antioxidant performance was surprising given the individual AP values at the use levels. Thus, there is a synergistic effect in the combination of Camellia sinensis (green tea) extract and a vitamin C derivative selected including ascorbyl glucoside.

### Example W/O Formulations

TABLE III
Example Sprays and Milks

| Component (INCI) | Function | Approx. wt. % |
|---|---|---|
| Example Main Ingredients | | |
| Water | Internal Phase | 10.0-<50.0 |
| Lauryl PEG-8 Dimethicone, Cetyl PEG/PPG- | Emulsifier | 0.5-5.0 |
| 10/1 Dimethicone | (HLB ≤ 6) | |
| Camellia sinensis (green tea) extract | Antioxidant | 0.05-0.5 |
| | (non-aqueous | |
| | dispersion) | |
| Camellia sinensis (green tea) extract | Antioxidant | 0.05-0.5 |
| | (aqueous dispersion) | |
| Additional Ingredients | | |
| Sodium Citrate, Sodium Chloride, | Chelating Agent, | Q.S. 100 |
| Phenoxyethanol, Ethylhexylglycerine, | Neutralizing Agent, | |
| Octocrylene, Homosalate, Ethylhexyl | Preservative System, | |
| Salicylate, Zinc Oxide, Titanium Dioxide, | Sunscreen, Solvent, | |
| Butyloctyl Salicylate, Caprylyl Glycol, Glycerin, | Skin Conditioner, | |
| C₁₂-C₁₅ Alkyl Benzoate, Dimethicone, | Additional | |
| Glyceryl Stearate, Polyhydroxystearic Acid, | Antioxidant, Film | |
| Caprylic/Capric Triglyceride, Panthenol, | Former, Colorant, | |
| Isododecane, Oil (coconut, soybean, | Absorbent, Fragrance | |
| sunflower), Butter (mango), Daucus Carota | | |
| Sativa (Carrot) Root Extract, vitamin C | | |
| derivative (ascorbyl glucoside), VP/Eicosene | | |
| Copolymer, Alcohol, mica (and) titanium | | |
| dioxide, mica (and) bismuth oxychloride (and) | | |
| iron oxides, Fragrance. | | |

Personal care compositions were formulated with the ingredients at the levels shown in Table 3. Example 11 (AP=76) included lauryl PEG-8 dimethicone and cetyl PEG/PPG-10/1 dimethicone, Camellia sinensis (green tea) extract in an amount from 0.1 wt. % to 0.15 wt. %, based on the total weight of the composition, and excluded thickener. Camellia sinensis (green tea) extract in crystalline dosage form was initially dispersed in ethanol to provide a ratio of extract to alcohol of about 1:10, based on the total weight of the dispersion. Additional ingredients included sodium chloride, phenoxyethanol, ethylhexylglycerin, zinc oxide, titanium dioxide caprylyl glycol, glycerin, C₁₂C₁₅ alkyl benzoate, dimethicone, glyceryl stearate, polyhydroxystearic acid, caprylic/capric triglyceride, panthenol, oil (coconut) Daucus Carota Sativa (Carrot) root extract, VP/Eicosene copolymer, mica (and) titanium dioxide, mica (and) bismuth oxychloride (and) iron oxides, and fragrance. Only mineral sunscreen filters were present in effective amounts for an SPF of ≥30.

Example 12 (AP=23) was similar to Example 11, except primarily that Camellia sinensis (green tea) extract was in aqueous dosage form (e.g., water, a diol, and additives) and further dispersed in ethanol before being added at the end to provide the same ratio of extract to alcohol of about 1:10, based on the total weight of the dispersion. In addition, Camellia 10 sinensis (green tea) extract was present in an amount from 0.15 wt. % to 3.0 wt. %, based on the total weight of the composition. Example 13 (AP=21) was similar to Example 12, except primarily that that Camellia sinensis (green tea) extract was present in an amount from 0.05 wt. % to 0.15 wt. %, based on the total weight of the composition, and included a vitamin C derivative (ascorbyl glucoside) to provide a ratio of Camellia sinensis (green tea) extract, ascorbyl glucoside, and alcohol of about 1:10:10, based on the total weight of the dispersion.

Example 14 (AP=97) included cetyl PEG/PPG-10/1 dimethicone, Camellia sinensis (green tea) extract in an amount from 0.1 wt. % to 0.15 wt. %, based on the total weight of the composition, and excluded thickener. Camellia sinensis (green tea) extract in crystalline dosage form was initially dispersed in isododecane to provide a ratio of extract to hydrocarbon of about 1:10, based on the total weight of the dispersion. Additional ingredients included sodium citrate, phenoxyethanol, octocrylene, homosalate, ethylhexyl salicylate, zinc oxide, butyloctyl salicylate, polyhydroxystearic acid, caprylic/capric triglyceride, isododecane, oils (soybean, sunflower), butter (mango), and Daucus Carota Sativa (Carrot) root extract. Organic and mineral sunscreen filters were present in effective amounts for an SPF of ≥50.

Example 15 (AP=35) was similar to Example 14, except primarily that Camellia sinensis (green tea) extract was in aqueous dosage form (e.g., water, a diol, and additives) and further dispersed in isododecane before being added at the end to provide a ratio of extract to hydrocarbon of about 1:3 to 1:4, based on the total weight of the dispersion. In addition, Camellia sinensis (green tea) extract and was present in an amount from 0.2 wt. % to 0.3 wt. %, based on the total weight of the composition.

Accordingly, although Examples 12-13 and 15 can provide acceptable antioxidant performance using Camellia sinensis (green tea) extract in aqueous dosage form, it may be preferable to use Camellia sinensis (green tea) extract in crystalline dosage form and most preferable to disperse the crystalline dosage form in a non-aqueous solvent for W/O emulsions (e.g., Examples 11 and 14). Moreover, it may be preferable to further disperse Camellia sinensis (green tea) extract in aqueous dosage form using a volatile solvent such as, for example, alcohol or hydrocarbon for W/O emulsions (e.g., Examples 12-13 and 15).

Comparative Example 4 (AP=10) was similar to Example 11, except primarily that the Camellia sinensis (green tea) extract that was dispersed in alcohol was then added to the water phase pre-emulsification. Comparative Example 5 (AP=2) was similar to Comparative Example 4, except primarily that the Camellia sinensis (green tea) extract was present in an amount of 80% less than the amount present in Comparative Example 4. Comparative Example 6 (AP=0) was similar to Example 11, except primarily that no Camellia sinensis (green tea) extract was present. Meanwhile, Comparative Example 7 (AP=7) was similar to Example 14, except primarily that the Camellia sinensis (green tea) extract that was dispersed in hydrocarbon was then added to the water phase pre-emulsification. Based on the antioxidant power observed, adding the Camellia sinensis (green tea) extract at the end of the manufacturing process when all phases are mixed (post-emulsification) under low temperature conditions may provide enhanced antioxidant performance in W/O emulsions.

### Example Anhydrous Formulations

TABLE IV
Example Sticks

| Component (INCI) | Function | Approx. wt. % |
|---|---|---|
| Example Main Ingredients | | |
| Ozokerite, Microcrystalline wax | Un-neutralized | 15.0-30.0 |
| | Wax Thickener | |
| Camellia sinensis (green tea) extract | Antioxidant | 0.0005-0.2 |
| | (crystalline) | |
| Additional Ingredients | | |
| Zinc Oxide, Isopropyl Myristate, Oil (avocado, | Sunscreen, Skin | Q.S. 100 |
| soybean), Silica, Daucus Carota Sativa (Carrot) | Conditioner, | |
| Root Extract, Water (≤5.0). | Additional Thickener, | |
| | Additional Antioxidant | |

Personal care compositions were formulated with the ingredients at the levels shown in Table 4. Example 16 (AP=147) included ozokerite and microcrystalline wax, and Camellia sinensis (green tea) extract in an amount from 0.05 wt. % to 0.1 wt. %, based on the total weight of the composition. Camellia sinensis (green tea) extract in crystalline dosage form was first added to a phase having isopropyl myristate and zinc oxide and then the phase was mixed with remaining phases, at relatively higher temperature conditions (e.g., 70° C.). Only mineral sunscreen filters were present in effective amounts for an SPF of ≥50.

Example 17 (AP=437) was similar to Example 16 except primarily that Camellia sinensis (green tea) extract was present in an amount from 0.1 wt. % to 0.2 wt. %, based on the total weight of the composition. Surprisingly, a two-fold increase from the already unexpectedly very high antioxidant performance may be achieved only with 50% more extract (notably, at relatively low use levels). Thus, Camellia sinensis (green tea) extract in crystalline dosage form may be used in an amount from 0.0005 wt. % to 0.2 wt. %, based on the total weight of the composition, for moderate to very high antioxidant performance at low levels.

TABLE V
Example Sprays

| Component (INCI) | Function | Approx. wt. % |
|---|---|---|
| Example Main Ingredients | | |
| Alcohol (Denatured) | Carrier | 35.0-80.0 |
| Camellia sinensis (green tea) extract | Antioxidant | 0.005-<0.1 |
| | (crystalline) | |
| Additional Ingredients | | |
| Octocrylene, Homosalate, Butyl | Sunscreen, Skin | Q.S. 100 |
| Methoxydibenzoylmethane, Ethylhexyl | Conditioner, | |
| Salicylate, Diisopropyl Adipate, Caprylyl Glycol, | Additional | |
| Glycerin, C₁₂-C₁₅ Alkyl Benzoate, Dimethicone, | Antioxidant, Film | |
| Caprylic/Capric Triglyceride, Panthenol, | Former, Fragrance | |
| Isododecane, Sea Salt, Oil (argan, soybean, | | |
| mineral), Butter (mango), Daucus Carota Sativa | | |
| (Carrot) Root Extract, vitamin E derivative | | |
| (Tocopheryl Acetate), vitamin C derivative | | |
| (Ascorbyl Palmitate, Tetrahexyldecyl | | |
| Ascorbate), vitamin B derivative (Niacinamide), | | |
| VA/Butyl Maleate/Isobornyl Acrylate | | |
| Copolymer, Aloe, Fragrance, extract (bamboo, | | |
| pea). | | |

Personal care compositions may be formulated with the ingredients at the levels shown in Table 4. Example 18 included alcohol in an amount from 55.0 wt. % to 75.0 wt. %, based on the total weight of the composition, and Camellia sinensis (green tea) extract in an amount from 0.005 wt. % to <0.1 wt. %, based on the total weight of the composition, and excluded thickener. Notably, Camellia sinensis (green tea) extract added in crystalline dosage form surprisingly provides very high antioxidant performance at the higher end of the use levels (AP=400, 500, etc.) within the listed range and therefore substantially lower levels across the listed range may be used for enhanced antioxidant performance. Additional ingredients included octocrylene, homosalate, butyl methoxydibenzoylmethane, diisopropyl adipate, caprylyl glycol, sea salt, dimethicone, isododecane, extract (bamboo, pea), Daucus Carota Sativa (Carrot) root extract, oil (soybean), VA/butyl maleate/isobornyl acrylate copolymer, fragrance, and if water, then <1.0 wt. %. Additional examples were formulated similar to Example 18, except that additional ingredients included octocrylene, homosalate, butyl methoxydibenzoylmethane, diisopropyl adipate, caprylyl glycol, dimethicone, caprylic/capric triglyceride, isododecane, Daucus Carota Sativa (Carrot) root extract, oil (soybean), vitamin C derivative (ascorbyl palmitate), VA/butyl maleate/isobornyl acrylate copolymer, fragrance, and if water, then <1.0 wt. %. Organic sunscreen filters were present in effective amounts for an SPF of ≥30.

Example 19 (AP=468) was tested as an alcohol-based concentrate which when aerosolized further included alcohol in an amount from 40.0 wt. % to 50.0 wt. %, based on the total weight of the composition, propellant (30 wt. %), and Camellia sinensis (green tea) extract in an amount from 0.05 wt. % to <0.1 wt. %, based on the total weight of the composition, and excluded thickener. Camellia sinensis (green tea) extract added was in crystalline dosage form. Additional ingredients included octocrylene, homosalate, butyl methoxydibenzoylmethane, ethylhexyl salicylate, diisopropyl adipate, caprylyl glycol, glycerin, C₁₂-C₁₅ alkyl benzoate, dimethicone isododecane, oils (argan, soybean), butter (mango), Daucus Carota Sativa (Carrot) root extract vitamin E derivative (tocopheryl acetate), vitamin C derivative (ascorbyl palmitate), VA/butyl maleate/isobornyl acrylate copolymer, fragrance, and if water, then <1.0 wt. %. Organic sunscreen filters were present in effective amounts for an SPF of ≥30. Additional examples were formulated similar to Example 19, except that additional ingredients included octocrylene, homosalate, butyl methoxydibenzoylmethane, ethylhexyl salicylate, caprylyl glycol, C₁₂-C₁₅ alkyl benzoate, dimethicone, isododecane, oils (argan, soybean), butter (mango), Daucus Carota Sativa (Carrot) root extract, vitamin E derivative (tocopheryl acetate), vitamin C derivative (ascorbyl palmitate), VA/butyl maleate/isobornyl acrylate copolymer, panthenol, fragrance, and if water, then <1.0 wt. %. Also, organic sunscreen filters were present in effective amounts for an SPF of ≥50.

Example 20 (AP=122) included alcohol in an amount from 40.0 wt. % to 50.0 wt. %, based on the total weight of the composition, propellant (25 wt. %), and Camellia sinensis (green tea) extract in an amount from 0.01 wt. % to 0.05 wt. %, based on the total weight of the composition. Camellia sinensis (green tea) extract added was in crystalline dosage form. Additional ingredients included octocrylene, homosalate, butyl methoxydibenzoylmethane, ethylhexyl salicylate, diisopropyl adipate, caprylyl glycol, glycerin, C₁₂C₁₅ alkyl benzoate, dimethicone, isododecane, oil (mineral), vitamin E derivative (tocopheryl acetate), vitamin C derivative (tetrahexyldecyl ascorbate), vitamin B derivative (niacinamide, a vitamin B3 derivative), VA/butyl maleate/isobornyl acrylate copolymer, aloe, and if water, then 1.0 wt. %. Organic sunscreen filters were present in effective amounts for an SPF of ≥50. Example 21 was similar to Example 20, except primarily that sunscreen filters were in effective amounts for an SPF of ≥50. It is understood that when a concentrate is to be aerosolized, levels of ingredients in the concentrate can be determined to account for a final concentration of the ingredients in the spray format and therefore the levels of the ingredients present in the concentrate are also within the scope of various aspects of embodiments.

Comparative Example 8, which exhibited no antioxidant performance (AP=9), was similar to Example 20, except primarily that no Camellia sinensis (green tea) extract was present. Surprisingly, when Camellia sinensis (green tea) extract was present with the vitamin C derivative, e.g., about a 1:16 ratio, over a ten-fold increase in antioxidant performance was observed in Example 20 (AP=122) even though the use level of the vitamin C derivative did not change. Example 22, which was similar to Example 20, except primarily that Camellia sinensis (green tea) extract was increased to provide a 1:10 ratio of extract to the vitamin C derivative, exhibited very high antioxidant performance (AP=229). This was surprising, given a modest increase of the Camellia sinensis (green tea) extract (on the order of hundredths of a wt. %). Thus, there is a synergistic effect in the combination of Camellia sinensis (green tea) extract and a vitamin C derivative including tetrahexyldecyl ascorbate.

TABLE VI
Example Oil Formulations

| Component (INCI) | Function | Approx. wt. % |
|---|---|---|
| Example Main Ingredients | | |
| Alcohol (Denatured) | Polar Carrier | 30.0-<50.0 |
| Diisopropyl Adipate, Caprylic/Capric | Non-Polar Carrier | 20.0-30.0 |
| Triglyceride, Isododecane | | |
| Camellia sinensis (green tea) extract | Antioxidant | 0.005-0.2 |
| | (crystalline) | |
| Additional Ingredients | | |
| Octocrylene, Homosalate, Butyl | Sunscreen, Skin | Q.S. 100 |
| Methoxydibenzoylmethane, Ethylhexyl | Conditioner, | |
| Salicylate, Caprylyl Glycol, Phenylisopropyl | Emulsifier, Additional | |
| Dimethicone, Polyglyceryl-3 Stearate/ | Antioxidant, Rheology | |
| Isostearate/Dimer Dilinoleate Crosspolymer, | Modifier, Fragrance | |
| Lauryl PEG-8 Dimethicone, Oils (argan, grape | | |
| seed, olive fruit), Vitamin E derivative | | |
| (Tocopheryl Acetate), Vitamin C derivative | | |
| (Ascorbyl Palmitate), Methyl Dihydroabietate | | |
| Extract Blend, Fragrance. | | |

Personal care compositions may be formulated with the ingredients at the levels shown in Table 6. Example 23 (AP=921) includes alcohol, oils, and Camellia sinensis (green tea) extract in an amount from 0.05 wt. % to 0.1 wt. %, based on the total weight of the composition. Notably, the Camellia sinensis (green tea) extract was first dispersed in the phase composed of the oils listed in Table 6 and then added to the bulk as part of that phase at the end. In this regard, the Camellia sinensis (green tea) extract may be dispersed in one or more of the oils in a ratio from 1:1 to 1:10. Moreover, Camellia sinensis (green tea) extract added in crystalline dosage form may surprisingly provide very high antioxidant performance at the higher end of the use levels and therefore substantially lower levels may be used for enhanced antioxidant performance. Additional ingredients included octocrylene, homosalate, butyl methoxydibenzoylmethane, ethylhexyl salicylate, caprylyl glycol, phenylisopropyl dimethicone, polyglyceryl-3 stearate/isostearate/dimer dilinoleate drosspolymer, lauryl PEG-8 dimethicone, oils (argan, grape seed, olive fruit), vitamin E derivative (tocopheryl acetate), vitamin C derivative (ascorbyl palmitate), methyl dihydroabietate, extract blend, fragrance, and if water, then <1.0 wt. %. Organic sunscreen filters were present in effective amounts for an SPF of ≥50. Example 24 was similar to Example 23, except primarily that sunscreen filters were in effective amounts for an SPF of 30.

### Example W/O Formulations

TABLE VII
Example Lotions (with Silicone Elastomer)

| Component (INCI) | Function | Approx. wt. % |
|---|---|---|
| Example Main Ingredients | | |
| Water | Internal Phase | 10.0-<50.0 |
| Lauryl Polyglyceral-3 | Emulsifier | 0.5-5.0 |
| Polydimethylsioxyethyl Dimethicone | (HLB ≤ 6) | |
| Dimethicone (and) Dimethicone/ | Emulsifying Silicone | 1.0-10.0 |
| Polyglycerin-3 Crosspolymer | Elastomer Gel Agent | |
| Acrylates/C₁₀₋₃₀ Alkyl | Neutralized Polymer | 0.01-1.0 |
| Acrylate Crosspolymer | Thickener | |
| Xanthan Gum | Un-neutralized | 0.1-1.0 |
| | Polymer Thickener | |
| Dimethicone (and) Disteardimonium | Mineral Thickener | 0.5-5.0 |
| Hectorite (and) Triethyl Citrate | (clay) | |
| Camellia sinensis (green tea) extract | Antioxidant | 0.005-0.5 |
| | (crystalline, aqueous | |
| | dispersion, and/or | |
| | non-aqueous | |
| | dispersion) | |
| Additional Ingredients | | |
| Dimethicone, Butyl Octyl Salicylate, | Carrier oil, Booster, | Q.S. 100 |
| Caprylic/Capric Triglycerides, | Neutralizing agent, | |
| Polyhydroxystearic acid, Zinc Oxide (and) | Buffer, Preservative | |
| Triethoxycaprylylsilane, Undecane (and) | system, Sunscreen, | |
| Tridecane, Butylene Glycol, Sodium Chloride, | Solvent, Skin | |
| Sodium Citrate, Phenoxyethanol, Silica | Conditioner, | |
| | Additional Thickener | |

Personal care compositions may be formulated with the ingredients at the levels shown in Table 7. Example 25 includes a polyglycerin/alkyl co-modified branched silicone emulsifier and an emulsifying silicone elastomer g U agent, a neutralized polymer thickener, an un-neutralized polymer thickener, a mineral clay thickener, and Camellia sinensis (green tea) extract in an amount from 0.005 wt. % to 0.5 wt. %, based on the total weight of the composition. Camellia sinensis (green tea) extract may be added to the examples shown at Table 8 in crystalline dosage form, in aqueous dosage form, in non-aqueous dosage form, or combinations thereof. Additional ingredients include carriers such as a silicone. Mineral sunscreen filters may be present in effective amounts for an SPF of ≥8 such as, e.g., 15, 50+.

### Example Gel Formulations

TABLE VIII
Example Anhydrous Gel

| Component (INCI) | Function | Approx. wt. % |
|---|---|---|
| Example Main Ingredients | | |
| Ethylexyl Palmitate, Isopropyl Myristate | Polar Carrier | 5.0-55.0 |
| Mineral Oil | Non-Polar Carrier | 15.0-40.0 |
| Thickener | Un-neutralized | 2.0-4.0 |
| | Thickener | |
| | (mineral) | |
| Camellia sinensis (green tea) extract | Antioxidant | 0.005-0.2 |
| | (crystalline and/or | |
| | non-aqueous | |
| | dispersion) | |
| Additional Ingredients | | |
| Octocrylene, Butyl Methoxydibenzoylmethane, | Sunscreen, Skin | Q.S. 100 |
| Oil (coconut), Vitamin E derivative (Tocopheryl | Conditioner, | |
| Acetate), Aloe, Extract (Fruit), Fragrance. | Emulsifier, Additional | |
| | Antioxidant, | |
| | Fragrance | |

Personal care compositions may be formulated with the ingredients at the levels shown in Table 8. Example 26 includes oil, thickener, and Camellia sinensis (green tea) extract in an amount from 0.005 wt. % to 0.2 wt. %, based on the total weight of the composition. The Camellia sinensis (green tea) extract may first be dispersed in the phase composed of the oils listed in Table 8 Sand then added to the bulk as part of that phase at the end. Additionally or alternatively, Camellia sinensis (green tea) extract may be dispersed in non-aqueous carrier and added in non-aqueous dosage form to the phase composed of the oils listed in Table 8. Additional ingredients include octocrylene, butyl methoxydibenzoylmethane, oil (coconut), vitamin E derivative (tocopheryl acetate), aloe, extract (fruit), and fragrance. Sunscreen filters may be present in amounts for an SPF of 8.

TABLE IX
Example Aqueous Gel

| Component (INCI) | Function | Approx. wt. % |
|---|---|---|
| Example Main Ingredients | | |
| Water | Polar Carrier | >80.0 |
| Polysorbate 20, Glyceryl Stearate (and) | Emulsifier | 0.5-1.5 |
| PEG-100 Stearate | (HLB ≥ 7) | |
| Xanthan Gum | Un-neutralized | 0.1-0.5 |
| | Polymer Thickener | |
| Carbomer, Acrylates/C₁₀₋₃₀ alkyl acrylate | Neutralized Polymer | 0.5-1.5 |
| crosspolymer | Thickener | |
| Camellia sinensis (green tea) extract | Antioxidant | 0.005-0.5 |
| | (aqueous dispersion) | |
| Additional Ingredients | | |
| Triethanolamine, Disodium EDTA, Sodium | pH Adjuster, Carrier, | Q.S. 100 |
| Hydroxide, Alcohol, Cetearyl Alcohol (and) | Co-Emulsifier, | |
| Cetyl Palmitate (and) Sorbitan Palmitate (and) | Sunscreen, | |
| Sorbitan Oleate, Benzophenone-4, | Preservative, | |
| Imidazolidinyl Urea, Benzyl Alcohol, | Preservative System, | |
| Iodopropynyl Butylcarbamate (and) PEG-4 | Skin Conditioner, Film | |
| laurate (and) PEG-4 dilaurate (and) PEG-4, | Former, Additional | |
| Phenoxyethanol (and) Ethylhexylglycerin, | Antioxidant, Extracts, | |
| Glycerin, Ethylhexyl Palmitate, Isododecane, | Amino Acids, | |
| Myristyl Lactate (and) Myristyl Alcohol, Butter | Fragrance, Colorant | |
| (shea), Panthenol, Polymethylsilsesquioxane, | | |
| vitamin C derivative (Sodium Ascorbyl | | |
| Phosphate), vitamin E derivative (Tocopheryl | | |
| Acetate), Aloe, Extract (fruit), Silk Amino | | |
| Acids, Fragrance, Colorant | | |

Personal care compositions may be formulated with the ingredients at the levels shown in Table 9. Example 27 includes polysorbate 20, carbomer, and Camellia sinensis (green tea) extract in an amount from 0.1 wt. % to 0.2 wt. %, based on the total weight of the composition. Camellia sinensis (green tea) extract may be added in aqueous dosage form (e.g., with water, a diol, and additives). Additional ingredients include triethanolamine, cetearyl alcohol (and) cetyl palmitate (and) sorbitan palmitate (and) sorbitan oleate, benzophenone-4, imidazolidinyl urea, iodopropynyl butylcarbamate (and) PEG-4 laurate (and) PEG-4 dilaurate (and) PEG-4, glycerin, alcohol denatured (<10 wt. %), aloe, fragrance, and colorant. Sunscreen filters may be present in amounts for an SPF of <8 such as, e.g., <2.

Example 28 includes a mixture of glyceryl stearate and PEG-100 stearate, xanthan gum, acrylates/C₁₀₋₃₀ alkyl acrylate crosspolymer, and Camellia sinensis (green tea) extract in an amount from 0.1 wt. % to 0.2 wt. %, based on the total weight of the composition. Camellia sinensis (green tea) extract may be added in aqueous dosage form (e.g., with water, a diol, and additives). Additional ingredients include disodium EDTA, sodium hydroxide, vitamin C derivative (sodium ascorbyl phosphate), extract (fruit), aloe, silk amino acids, glycerin, ethylhexyl palmitate, isododecane, myristyl lactate (and) myristyl alcohol, butter (shea), polymethylsilsesquioxane, benzophenone-4, vitamin E derivative (tocopheryl acetate), panthenol, benzyl alcohol, phenoxyethanol (and) ethylhexylglycerin, and fragrance. In this example, sunscreen filters are present in amounts for an SPF of <8 such as, e.g., <2, etc.

It is understood that the examples above include aspects of non-limiting embodiments, and that listed ingredients or respective levels or ratios may be modified based on various formulation criteria such as, without limitation, photoprotection, sensory profile, antioxidant power, formulation format, and so on. For instance, Example 23 may be modified to replace any or all of the polar and/or non-polar oil carriers listed in Table 6 with one or more non-polar carriers (e.g., mineral oil, silicone oil, etc.). Additionally or alternatively, the polar alcohol carrier may be replaced in part or in whole (e.g., 1:1 ratio, etc.) with one or more of the non-polar carriers listed in Table 6, with one or more carriers described herein, or the like (e.g., esters, etc.). Thus, the relatively light sensory profile imparted by Example 23 may be modified to a relatively dry and low-slip formulation. Additionally or alternatively, Camellia sinensis (green tea) extract may be added to any example formulation in crystalline dosage form, in aqueous dosage form (e.g., water, a diol, and additives), alone or further dispersed in a non-aqueous carrier such as in isododecane or silicone carrier, in non-aqueous dosage form (initially dispersed in a non-aqueous carrier such as ethanol and/or hydrocarbon), or combinations of dosage forms may be used, in any desired use level.

### Additional Notes and Examples

Additional Examples include a personal care composition, a personal care product (consumer packaged product) including the personal care composition, a method of making the personal care composition and/or the personal care product, a method of using the personal care composition and/or the personal care product, or combinations thereof.

Additional Example 1 may include a personal care composition comprising water present in an amount from 50.0 wt. % to 90.0 wt. %, based on the total weight of the composition, an emulsifier having a hydrophilic-lipophilic balance (HLB) value≥7, a polymer thickener present in an amount from 0.1 wt. % to 1.0 wt. %, based on the total weight of the composition, and Camellia sinensis (green tea) extract present in an amount from 0.005 wt. % to 0.2 wt. %, based on the total weight of the composition.

Additional Example 2 may include the personal care composition of Additional Example 1, wherein the Camellia sinensis (green tea) extract is in an aqueous dosage form.

Additional Example 3 may include the personal care composition of any of Additional Examples 1-2, further including a sunscreen filter.

Additional Example 4 may include the personal care composition of any of Additional Examples 1-3, wherein the polymer thickener includes an un-neutralized polymer thickener present in an amount from 0.1 wt. % to 1.0 wt. %, based on the total weight of the composition, and wherein Camellia sinensis (green tea) extract is present in an amount from 0.005 wt. % to 0.2 wt. %, based on the total weight of the composition.

Additional Example 5 may include the personal care composition of any of Additional Examples 1-4, wherein the sunscreen filter includes an organic sunscreen filter, a mineral sunscreen filter, or combinations thereof, wherein the un-neutralized polymer thickener includes xanthan gum, and wherein the Camellia sinensis (green tea) extract is present in an amount from 0.1 wt. % to 0.2 wt. %, based on the total weight of the composition.

Additional Example 6 may include the personal care composition of any of Additional Examples 1-5, wherein the un-neutralized polymer thickener includes xanthan gum, wherein the Camellia sinensis (green tea) extract is present in an amount from 0.1 wt. % to 0.2 wt. %, based on the total weight of the composition, and wherein the personal care composition excludes any sunscreen filter.

Additional Example 7 may include the personal care composition of any of Additional Examples 1-6, wherein the polymer thickener includes a neutralized polymer thickener present in an amount from 0.1 wt. % to 1.0 wt. %, based on the total weight of the composition, and wherein the Camellia sinensis (green tea) extract is present in an amount from 0.04 wt. % to 0.2 wt. %, based on the total weight of the composition.

Additional Example 8 may include the personal care composition of any of Additional Examples 1-7, wherein the sunscreen filter comprises an organic sunscreen filter, wherein the neutralized polymer thickener includes acrylates/C₁₀₋₃₀ alkyl acrylate crosspolymer, and wherein the Camellia sinensis (green tea) extract is present in an amount from 0.1 wt. % to 0.2 wt. %, based on the total weight of the composition.

Additional Example 9 may include the personal care composition of any of Additional Examples 1-8, further including a vitamin C derivative, wherein a ratio of the Camellia sinensis (green tea) extract to the vitamin C derivative is from 1:1 to 1:20.

Additional Example 10 may include the personal care composition of any of Additional Examples 1-9, wherein the neutralized polymer thickener includes carbomer, wherein the Camellia sinensis (green tea) extract is present in an amount from 0.04 wt. % to <0.1 wt. %, based on the total weight of the composition, and wherein the vitamin C derivative includes ascorbyl glucoside.

Additional Example 11 may include the personal care composition of any of Additional Examples 1-10, wherein the neutralized polymer thickener includes carbomer, and wherein the Camellia sinensis (green tea) extract is present in an amount from 0.1 wt. % to 0.2 wt. %, based on the total weight of the composition.

Additional Example 12 may include a personal care composition comprising water present in an amount from 10.0 wt. % to <50.0 wt. %, based on the total weight of the composition, an emulsifier having a hydrophilic-lipophilic balance (HLB) value≤6, and Camellia sinensis (green tea) extract present in an amount from 0.05 wt. % to 0.5 wt. %, based on the total weight of the composition.

Additional Example 13 may include the personal care composition of Additional Example 12, further including a sunscreen filter.

Additional Example 14 may include the personal care composition of any of Additional Examples 12-13, wherein the Camellia sinensis (green tea) extract is in a non-aqueous dosage form, and wherein the Camellia sinensis (green tea) extract is present in an amount from 0.05 wt. % to 0.2 wt. %, based on the total weight of the composition.

Additional Example 15 may include the personal care composition of any of Additional Examples 12-14, wherein the Camellia sinensis (green tea) extract is initially dispersed in a C₂-C₄ alcohol, a hydrocarbon, or combinations thereof, and wherein the Camellia sinensis (green tea) leaf extract is present in an amount from 0.01 wt. % to 0.15 wt. %, based on the total weight of the composition.

Additional Example 16 may include the personal care composition of any of Additional Examples 12-15, wherein the Camellia sinensis (green tea) extract is in an aqueous dosage form.

Additional Example 17 may include the personal care composition of any of Additional Examples 12-16, wherein the Camellia sinensis (green tea) extract in aqueous dosage form is further dispersed in a C₂-C₄ alcohol, and wherein the Camellia sinensis (green tea) extract is present in an amount from 0.05 wt. % to 0.3 wt. %, based on the total weight of the composition.

Additional Example 18 may include the personal care composition of any of Additional Examples 12-17, further including vitamin C or a derivative thereof.

Additional Example 19 may include the personal care composition of any of Additional Examples 12-18, wherein the Camellia sinensis (green tea) extract in aqueous dosage form is further dispersed in a hydrocarbon, and wherein the Camellia sinensis (green tea) extract is present in an amount from 0.2 wt. % to 0.3 wt. %, based on the total weight of the composition.

Additional Example 20 may include the personal care composition of any of Additional Examples 12-19, wherein the emulsifier includes a silicone emulsifier, and wherein the personal care composition further includes one or more of a silicone elastomer gel agent, a mineral thickener, an un-neutralized thickener, or a neutralized thickener.

Additional Example 21 may include a personal care composition comprising water present in an amount ≤5.0 wt. %, based on the total weight of the composition, a non-aqueous carrier present in an amount from 20.0 wt. % to 80.0 wt. %, based on the total weight of the composition, and Camellia sinensis (green tea) extract present in an amount from 0.005 wt. % to 0.2 wt. %, based on the total weight of the composition.

Additional Example 22 may include the personal care composition of Additional Example 21, wherein the Camellia sinensis (green tea) extract is in a crystalline dosage form, non-aqueous dosage form, or combinations thereof.

Additional Example 23 may include the personal care composition of any of Additional Examples 21-22, further including a sunscreen filter.

Additional Example 24 may include the personal care composition of any of Additional Examples 21-23, wherein the non-aqueous carrier includes a C₂-C₄ alcohol solvent present in an amount from 35.0 wt. % to 80.0 wt. %, based on the total weight of the composition, and wherein the Camellia sinensis (green tea) extract is present in an amount from 0.005 wt. % to <0.1 wt. %, based on the total weight of the composition.

Additional Example 25 may include the personal care composition of any of Additional Examples 21-24, wherein the Camellia sinensis (green tea) extract is present in an amount from 0.05 wt. % to <0.1 wt. %, based on the total weight of the composition, and wherein the personal care composition further includes a propellant.

Additional Example 26 may include the personal care composition of any of Additional Examples 21-25, wherein the Camellia sinensis (green tea) extract is present in an amount from 0.01 wt. % to 0.05 wt. %, based on the total weight of the composition.

Additional Example 27 may include the personal care composition of any of Additional Examples 21-26, further including a vitamin C derivative comprising tetrahexyldecyl ascorbate.

Additional Example 28 may include the personal care composition of any of Additional Examples 21-27, wherein the non-aqueous carrier includes a non-polar carrier present in an amount from 20.0 wt. % to 80.0 wt. %, based on the total weight of the composition, and wherein the Camellia sinensis (green tea) extract is present in an amount from 0.005 wt. % to 0.2 wt. %, based on the total weight of the composition.

Additional Example 29 may include the personal care composition of any of Additional Examples 21-28, wherein the Camellia sinensis (green tea) extract is present in an amount from 0.05 wt. % to 1.0 wt. %, based on the total weight of the composition.

Additional Example 30 may include the personal care composition of any of Additional Examples 21-29, wherein the non-polar carrier is selected from an ester, an ether, a fatty alcohol, a silicone, a hydrocarbon oil, a polyol, or combinations thereof.

Additional Example 31 may include the personal care composition of any of Additional Examples 21-30, wherein the silicone includes a polyalkylsiloxane, an alkyl substituted dimethicone, a dimethiconol, a polyalkylaryl siloxane, or combinations thereof, and wherein the personal care composition further includes a silicone emulsifier, a silicone elastomer gel agent, or combinations thereof.

Additional Example 32 may include the personal care composition of any of Additional Examples 21-31, wherein the hydrocarbon oil includes mineral oil, a polyolefin, a paraffin, an isoparaffin, a vegetable oil, or combinations thereof.

Additional Example 33 may include the personal care composition of any of Additional Examples 21-32, further including a C₂-C₄ alcohol solvent.

Additional Example 34 may include the personal care composition of any of Additional Examples 21-33, wherein the non-aqueous carrier includes a polar carrier present in an amount from 30.0 wt. % to 70.0 wt. %, based on the total weight of the composition, and wherein the personal care composition further includes an un-neutralized wax thickener present in an amount from 15.0 wt. % to 30.0 wt. %, based on the total weight of the composition.

Additional Example 35 may include the personal care composition of any of Additional Examples 21-34, wherein the un-neutralized wax thickener is selected from an animal wax, a vegetable wax, a mineral wax, a petroleum wax, a silicone wax, or combinations thereof.

Additional Example 36 may include the personal care composition of any of Additional Examples 21-35, wherein the un-neutralized wax thickener is selected from an alkyl silicone, alkyl trimethylsilane, beeswax, behenyl behenate, cerotyl dimethicone, candelilla wax, carnauba wax, synthetic carnauba, PEG-12 carnauba, myrica wax, rice bran wax, soy wax, ceresin, hydrogenated microcrystalline wax, jojoba wax, microcrystalline wax, lanolin wax, ozokerite, paraffin, synthetic paraffin, cetyl esters, C₂₀₋₄₀ alkyl behenate, cetyl palmitate, stearyl palmitate, lauryl behenate, cetyl octanoate, cetyl ricinoleate, cetyl stearate, decyl oleate, myristyl stearate, lauryl stearate, octyldodecyl stearate, octyldodecyl stearoyl stearate, oleyl arachidate, oleyl stearate, tridecyl stearate, pentaerythrityl tetrabehenate, pentaerythrityl distearate, polyethylene, hydrogenated cottonseed oil, hydrogenated vegetable oil, hydrogenated jojoba oil, hydrogenated palm oil, hydrogenated palm kernel oil, hydrogenated olive oil, polyamides, C₃₀-C₆₀ fatty alcohols, polypropylene, polybutene, zinc stearate, stearyl palmitate, stearyl behenate, shellac wax, glycol montanate, C₂₀-C₄₀ alkyl hydroxystearyl stearate, tribehenin, or combinations thereof.

Additional Example 37 may include the personal care composition of any of Additional Examples 21-36, wherein the Camellia sinensis (green tea) extract is present in an amount from 0.05 wt. % to 0.2 wt. %, based on the total weight of the composition, and wherein the un-neutralized wax thickener is present in an amount from 18.0 wt. % to 25.0 wt. %, based on the total weight of the composition.

Additional Example 38 may include the personal care composition of any of Additional Examples 21-37, wherein the polar carrier includes an ester of a C₂-C₄ alcohol and a fatty acid.

Additional Example 39 may include the personal care composition of any of Additional Examples 21-38 wherein the personal care composition is in the form of a spray, a gel, or a stick.

Additional Example 40 may include the personal care composition of any of Additional Examples 1-39, further comprising one or more film formers, anti-aging actives, insect repellants, amino acids, colorants, pigments, dyes, opacifiers, pH adjusters, fragrances, fillers, vitamins and its derivatives, preservatives, preservative boosters, chelating agents, stabilizers, sphingolipids, antioxidants, minerals, keratolytics, hormonal compounds, analgesics, anti-allergenic agents, H1 or H2 antihistamines, anti-inflammatory agents, anti-irritants, anti-neoplastics, immune system boosting agents, immune system suppressing agents, anti-acne agents, anesthetics, antiseptics, skin cooling compounds, skin protectants, skin penetration enhancers, exfoliants, staining agents, depigmenting agents, hypopigmenting agents, pharmaceutical agents, spherical powders, extracts, alpha and beta hydroxy acids, retinol and its derivatives, elastomers, propellants, or combinations thereof.

Additional Example 41 may include a method to manufacture the personal care composition of any of Additional Examples 1-39, a personal care product including the personal care composition of any of Additional Examples 1-39, or combinations thereof.

Additional Example 42 may include the method of Additional Example 41, comprising mixing two or more ingredients of the personal care composition of any of Additional Examples 1-39, combining the personal care composition of any of Additional Examples 1-39 with a product package, or combinations thereof.

Additional Example 43 may include a method to use the personal care composition of any of Additional Examples 1-39, a personal care product including the personal care composition of any of Additional Examples 1-39, or combinations thereof.

Additional Example 44 may include the method of Additional Example 43, comprising applying the personal care composition of any of Additional Examples 1-39 onto a keratinous or hirsute substrate.

Additional Example 45 may include the method of any of Additional Examples 43-44, comprising applying the personal care composition from the personal care product.

In this disclosure and the following claims, the word “or” indicates an inclusive list such that, for example, the list of X, Y, or Z means X or Y or Z or XY or XZ or YZ or XYZ. Similarly, the list of one or more of X, Y, or Z or a list of X, Y, Z, or combinations thereof means X or Y or Z or XY or XZ or YZ or XYZ. Also the phrase “based on” is not used to represent a closed set of conditions. For example, a step that is described as “based on condition A” may be based on both condition A and condition B. In other words, the phrase “based on” shall be construed to mean “based at least in part on.” Also, the words “a” or “an” indicate “at least one.”

Accordingly, while the present disclosure has been particularly described, in conjunction with specific preferred embodiments, it is evident that many alternatives, modifications, and variations will be apparent to those skilled in the art in light of the foregoing description. It is therefore contemplated that the appended claims will embrace any such alternatives, modifications, and variations as falling within the true scope and spirit of the present disclosure.

1-40. (canceled)

41. A personal care composition comprising:
a sunscreen filter;
water present in an amount ≤5.0 wt. %, based on the total weight of the composition;
a non-aqueous carrier present in an amount from 20.0 wt. % to 80.0 wt. %, based on the total weight of the composition; and
Camellia sinensis (green tea) extract present in an amount from 0.005 wt. % to 0.2 wt. %, based on the total weight of the composition,
wherein the Camellia sinensis (green tea) extract is dispersed in the non-aqueous carrier in a crystalline dosage form or is dispersed in a non-aqueous solvent to provide a pre-mix in a non-aqueous dosage form, and
wherein the personal care composition is in the form of a spray, a stick, or a gel.

42. The personal care composition of claim 41, wherein the non-aqueous carrier includes a C₂₋₄ alcohol solvent present in an amount from 35.0 wt. % to 80.0 wt. %, based on the total weight of the composition, and wherein the Camellia sinensis (green tea) extract is present in an amount from 0.005 wt. % to <0.1 wt. %, based on the total weight of the composition.

43. The personal care composition of claim 42, wherein the Camellia sinensis (green tea) extract is present in an amount from 0.05 wt. % to <0.1 wt. %, based on the total weight of the composition, and wherein the personal care composition further includes a propellant.

44. The personal care composition of claim 42, wherein the Camellia sinensis (green tea) extract is present in an amount from 0.01 wt. % to 0.05 wt. %, based on the total weight of the composition.

45. The personal care composition of claim 44, further including a vitamin C derivative comprising tetrahexyldecyl ascorbate.

46. The personal care composition of claim 41, wherein the non-aqueous carrier includes a non-polar carrier present in an amount from 20.0 wt. % to 80.0 wt. %, based on the total weight of the composition, and wherein the Camellia sinensis (green tea) extract is present in an amount from 0.005 wt. % to 0.2 wt. %, based on the total weight of the composition.

47. The personal care composition of claim 46, wherein the Camellia sinensis (green tea) extract is present in an amount from 0.05 wt. % to 1.0 wt. %, based on the total weight of the composition.

48. The personal care composition of claim 46, wherein the non-polar carrier is selected from an ester, an ether, a fatty alcohol, a silicone, a hydrocarbon oil, a polyol, or combinations thereof.

49. The personal care composition of claim 48, wherein the silicone includes a polyalkylsiloxane, an alkyl substituted dimethicone, a dimethiconol, a polyalkylaryl siloxane, or combinations thereof, and wherein the personal care composition further includes a silicone emulsifier, a silicone elastomer gel agent, or combinations thereof.

50. The personal care composition of claim 48, wherein the hydrocarbon oil includes mineral oil, a polyolefin, a paraffin, an isoparaffin, a vegetable oil, or combinations thereof.

51. The personal care composition of claim 48, further including a C₂-C₄ alcohol solvent.

52. The personal care composition of claim 41, wherein the non-aqueous carrier includes a polar carrier present in an amount from 30.0 wt. % to 70.0 wt. %, based on the total weight of the composition, and wherein the personal care composition further includes an un-neutralized wax thickener present in an amount from 15.0 wt. % to 30.0 wt. %, based on the total weight of the composition.

53. The personal care composition of claim 52, wherein the un-neutralized wax thickener is selected from an animal wax, a vegetable wax, a mineral wax, a petroleum wax, a silicone wax, or combinations thereof.

54. The personal care composition of claim 53, wherein the un-neutralized wax thickener is selected from an alkyl silicone, alkyl trimethylsilane, beeswax, behenyl behenate, cerotyl dimethicone, candelilla wax, carnauba wax, synthetic carnauba, PEG-12 carnauba, myrica wax, rice bran wax, soy wax, ceresin, hydrogenated microcrystalline wax, jojoba wax, microcrystalline wax, lanolin wax, ozokerite, paraffin, synthetic paraffin, cetyl esters, C₂₀-C₄₀ alkyl behenate, cetyl palmitate, stearyl palmitate, lauryl behenate, cetyl octanoate, cetyl ricinoleate, cetyl stearate, decyl oleate, myristyl stearate, lauryl stearate, octyldodecyl stearate, octyldodecyl stearoyl stearate, oleyl arachidate, oleyl stearate, tridecyl stearate, pentaerythrityl tetrabehenate, pentaerythrityl distearate, polyethylene, hydrogenated cottonseed oil, hydrogenated vegetable oil, hydrogenated jojoba oil, hydrogenated palm oil, hydrogenated palm kernel oil, hydrogenated olive oil, polyamides, C₃₀-C₆₀ fatty alcohols, polypropylene, polybutene, zinc stearate, stearyl palmitate, stearyl behenate, shellac wax, glycol montanate, C₂₀₋₄₀ alkyl hydroxystearyl stearate, tribehenin, or combinations thereof.

55. The personal care composition of claim 53, wherein the Camellia sinensis (green tea) extract is present in an amount from 0.05 wt. % to 0.2 wt. %, based on the total weight of the composition, and wherein the un-neutralized wax thickener is present in an amount from 18.0 wt. % to 25.0 wt. %, based on the total weight of the composition.

56. The personal care composition of claim 41, wherein the polar carrier includes an ester of a C₂-C₄ alcohol and a fatty acid.

57. The personal care composition of claim 41, wherein the personal care composition is in the form of a stick.

58. The personal care composition of claim 41, wherein the personal care composition is in the form of a spray.

59. The personal care composition of claim 41, further comprising one or more film formers, anti-aging actives, insect repellants, amino acids, colorants, pigments, dyes, opacifiers, pH adjusters, fragrances, fillers, vitamins and its derivatives, preservatives, preservative boosters, chelating agents, stabilizers, sphingolipids, antioxidants, minerals, keratolytics, hormonal compounds, analgesics, anti-allergenic agents, H1 or H2 antihistamines, anti-inflammatory agents, anti-irritants, anti-neoplastics, immune system boosting agents, immune system suppressing agents, anti-acne agents, anesthetics, antiseptics, skin cooling compounds, skin protectants, skin penetration enhancers, exfoliants, staining agents, depigmenting agents, hypopigmenting agents, pharmaceutical agents, spherical powders, extracts, alpha and beta hydroxy acids, retinol and its derivatives, elastomers, propellants, or combinations thereof.
